(19) United States
(12) Patent Application Publication
(10) Pub. No.: US 2012/0295915 A1
(43) Pub. Date: Nov. 22, 2012

(54) AZABENZIMIDAZOLES AS FATTY ACID SYNTHASE INHIBITORS

(76) Inventors: Amita M. Chaudhari, Collegeville, PA (US); Jason Hallman, Cary, NC (US); Christopher P. Laudeman, Durham, NC (US); David Lee Musso, Raleigh, NC (US); Cynthia A. Parrish, Collegeville, PA (US)

(21) Appl. No.: 13/511,767

(22) PCT Filed: Nov. 22, 2010
(86) PCT No.: PCT/US2010/057594

§ 371 Date: May 24, 2012

Related U.S. Application Data
(60) Provisional application No. 61/263,889, filed on Nov. 24, 2009.
(60) Provisional application No. 61/286,890, filed on Dec. 16, 2009.

(51) Int. Cl. C07D 401/14; A61P 35/02; A61K 31/52; A61P 35/00; A61K 31/437; C07D 473/40

(52) U.S. Cl. 514/252.16; 546/118; 514/303; 544/277; 514/263.24

(57) ABSTRACT

Disclosed are compounds having Formula (I),

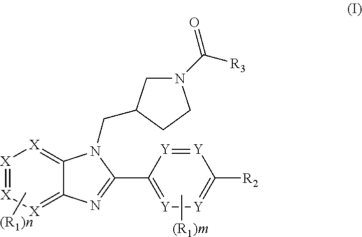

wherein R¹, R², R³, R⁴, Ra, Rb, X, Y, m, and n are defined herein and methods of using the same.

### FIELD OF INVENTION

This invention relates to novel azabenzimidazoles which are inhibitors of fatty acid synthase (FAS), to pharmaceutical compositions containing them, to processes for their preparation, and to their use in therapy for the treatment of cancers.

### BACKGROUND

Fatty acids have an essential role in a variety of cellular processes including building blocks for membranes, anchors for targeting membrane proteins, precursors in the synthesis of lipid second messengers and as a medium to store energy, Menendez J S and Lupu R, Fatty acid synthase and the lipogenic phenotype in cancer pathogenesis, Nature Reviews Cancer, 7: 763-777 (2007). Fatty acids can either be obtained from the diet or can be synthesized de novo from carbohydrate precursors. The biosynthesis of the latter is catalyzed by the muliti-functional homodimeric FAS. FAS synthesizes long chain fatty acids by using acetyl-CoA as a primer and Malonyl Co-A as a 2 carbon donor, and NADPH as a reducing equivalents (Wakil S J, Lipids, Structure and function of animal fatty acid synthase, 39: 1045-1053 (2004), Asturias F J et al., Structure and molecular organization of mammalian fatty acid synthase, Nature Struct. Mol. Biol. 12:225-232 (2005), Maier T, et al., Architecture of Mammalian Fatty Acid Synthase at 4.5 Å Resolution, Science 311:1258-1262 (2006).

De novo fatty acid synthesis is active during embryogenesis and in fetal lungs where fatty acids are used for the production of lung surfactant. In adults, most normal human tissues preferentially acquire fatty acids from the diet. Therefore the level of de novo lipogensis and expression of liopogenic enzymes is low, Weiss L, et al., Fatty-acid biosynthesis in man, a pathway of minor importance. Purification, optimal assay conditions, and organ distribution of fatty-acid synthase. Biological Chemistry Hoppe-Seyler 367(9):905-912 (1986). In contrast, many tumors have high rates of de novo fatty acid synthesis Medes G, et al., Metabolism of Neoplastic Tissue. IV. A Study of Lipid Synthesis in Neoplastic Tissue Slices in Vitro, Can Res, 13:27-29, (1953). FAS has now been shown to be overexpressed in numerous cancer types including prostate, ovary, colon, endometrium lung, bladder, stomach and kidney Kuhajda F P, Fatty-acid synthase and human cancer: new perspectives on its role in tumor biology, Nutrition; 16:202-208 (2000). This differential expression and function of FAS in tumors and normal cells provide an approach for cancer therapy with the potential of a substantial therapeutic window.

Pharmacological and small interference RNA mediated inhibition of FAS has demonstrated a preferential inhibition of cancer cell proliferation. Additionally these inhibitors induce apoptosis in cancers cells in vitro and retard growth in human tumors in murine xenograft models in vivo, Menendez J S and Lupu R, Nature Reviews Cancer, 7: 763-777 (2007). Based upon these findings, FAS is considered a major potential target of antineoplastic intervention.

### SUMMARY OF THE INVENTION

This invention relates to compound of the Formula (I), as shown below

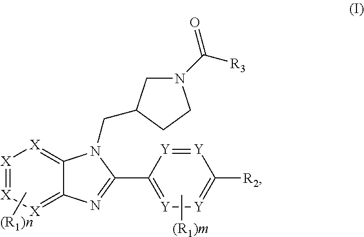

wherein,
each R₁ is independently selected from the group consisting of: C1-6alkyl, alkoxy, hydroxyl, halogen, amino, substituted amino, alkylsulfonyl, cyano, heterocycloalkyl and —C(O)NRₐR_{b},
in which Rₐ and R_{b} are hydrogen, C1-6alkyl, C3-7cycloalkyl, or together Rₐ and R_{b} form a C3-7heterocycloalkyl;
R₂ is selected from the group consisting of: aryl and heteroaryl, in which adjacent substituents in said aryl or heteroaryl together may form an additional five or six membered ring which contains 0-2 hetero atoms;
R₃ is selected from the group consisting of: amino, alkylamino, dialkylamino, —OC1-6alkyl, C1-6alkyl and C3-7cycloalkyl;
R₄ is selected from the group consisting of: C1-6alkyl, alkoxy, hydroxyl, and halogen;

### Y and X are C or N;

n is 0-3
m is 0-4;
or a pharmaceutically acceptable salt thereof;
with the proviso that at least one but no more than two X's are N and at least two Y's are C.

This invention also relates to pharmaceutical compositions, which comprise compounds of Formula (I) and pharmaceutically acceptable carriers.

This invention also relates to methods of treating cancer which comprises administering an effective amount of a compound of formula (I) to a human in need thereof.

This invention also relates to methods of treating cancer which comprise co-administering an compound of Formula (I) and a second compound to a human in need thereof.

### DETAILED DESCRIPTION OF THE INVENTION

This invention also relates to compound of the Formula (I)(A), as shown below

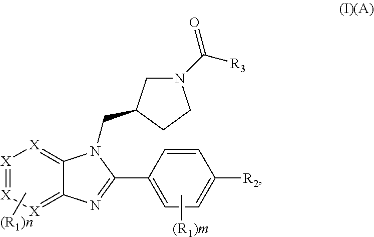

wherein,
each R₁ is independently selected from the group consisting of: C1-6alkyl, alkoxy, hydroxyl, halogen, amino, alkylamino, dialkylamino, cyano, alkylsulfonyl, heterocycloalkyl and —C(O)NRₐR_{b}, in which Rₐ and R_{b} are hydrogen, C1-6alkyl, C3-7cycloalkyl, or together Rₐ and R_{b} form a C3-7heterocycloalkyl;
R₂ is selected from the group consisting of: aryl and heteroaryl, in which adjacent substituents in said aryl or heteroaryl together may form an additional five or six membered ring which contains 0-2 hetero atoms;
R₃ is selected from the group consisting of: amino, alkylamino, dialkylamino, —OC1-6alkyl, C1-6alkyl and C3-7cycloalkyl;
R₄ is selected from the group consisting of: C1-6alkyl, alkoxy, hydroxyl, and halogen;

### X is C or N;

n is 0-3
m is 0-4;
or a pharmaceutically acceptable salt thereof;
with the proviso that at least one but no more than two X's are N.

This invention also relates to compounds of Formula (I)(A), wherein R₃ is cyclopropyl.

This invention also relates to compounds of Formula (I)(A), wherein n is 0-2 and m is 0.

This invention also relates to compounds of Formula (I)(A), wherein n is 0-1 and m is 1

This invention also relates to compounds of Formula (I)(A), wherein R₁ is halogen, C1-3alkyl, amino, or alkylaminoas defined above.

This invention also relates to compounds of Formula (I)(A), wherein R₂ is heteroaryl.

This invention also relates to compounds of Formula (I)(A), wherein R₂ is aryl.

This invention also relates to compounds of Formula (I)(A), wherein R₂ is pyrrolopyridinyl, imidazopyridinyl, benzimidazolyl, benzothiazolyl, benzofuranyl or indolyl.

This invention also relates to the following compounds:
4′-(1-{[(3S)-1-(cyclopropylcarbonyl)-3-pyrrolidinyl]methyl}-1H-imidazo[4,5-b]pyridin-2-yl)-3-biphenylol,
1-{[(3S)-1-(cyclopropylcarbonyl)-3-pyrrolidinyl]methyl}-2-(3′-methyl-4-biphenylyl)-1H-imidazo[4,5-b]pyridine,
1-{[(3S)-1-(cyclopropylcarbonyl)-3-pyrrolidinyl]methyl}-2-(2′,4′-dimethyl-4-biphenylyl)-1H-imidazo[4,5-b]pyridine,
2-[2′-chloro-4′-(methyloxy)-4-biphenylyl]-1-{[(3S)-1-(cyclopropylcarbonyl)-3-pyrrolidinyl]methyl}-1H-imidazo[4,5-b]pyridine,
1-{[(3S)-1-(cyclopropylcarbonyl)-3-pyrrolidinyl]methyl}-2-[4-(1H-indazol-5-yl)phenyl]-1H-imidazo[4,5-b]pyridine,
1-{[(3S)-1-(cyclopropylcarbonyl)-3-pyrrolidinyl]methyl}-2-[4-(1H-indol-6-yl)phenyl]-1H-imidazo[4,5-b]pyridine,
1-{[(3S)-1-(cyclopropylcarbonyl)-3-pyrrolidinyl]methyl}-2-(4′-methyl-4-biphenylyl)-1H-imidazo[4,5-b]pyridine,
1-{[(3S)-1-(cyclopropylcarbonyl)-3-pyrrolidinyl]methyl}-2-(2′,4′-dichloro-4-biphenylyl)-1H-imidazo[4,5-b]pyridine,
1-{[(3S)-1-(cyclopropylcarbonyl)-3-pyrrolidinyl]methyl}-2-(4′-fluoro-3′-methyl-4-biphenylyl)-1H-imidazo[4,5-b]pyridine,
2-(4-biphenylyl)-1-{[(3S)-1-(cyclopropylcarbonyl)-3-pyrrolidinyl]methyl}-1H-imidazo[4,5-b]pyridine,
2-[4-(1-benzofuran-5-yl)phenyl]-1-{[(3S)-1-(cyclopropylcarbonyl)-3-pyrrolidinyl]methyl}-1H-imidazo[4,5-b]pyridine,
1-{[(3S)-1-(cyclopropylcarbonyl)-3-pyrrolidinyl]methyl}-2-(4′-fluoro-4-biphenylyl)-1H-imidazo[4,5-b]pyridine,
2-(4-biphenylyl)-1-{[1-(cyclopropylcarbonyl)-3-pyrrolidinyl]methyl}-1H-imidazo[4,5-c]pyridine,
1-{[1-(cyclopropylcarbonyl)-3-pyrrolidinyl]methyl}-2-[4-(1H-indazol-5-yl)phenyl]-1H-imidazo[4,5-c]pyridine,
1-{[(3S)-1-acetyl-3-pyrrolidinyl]methyl}-2-(4-biphenylyl)-1H-imidazo[4,5-c]pyridine,
2-(4-biphenylyl)-1-{[(3S)-1-propanoyl-3-pyrrolidinyl]methyl}-1H-imidazo[4,5-c]pyridine,
2-(4-biphenylyl)-1-{[(3S)-1-butanoyl-3-pyrrolidinyl]methyl}-1H-imidazo[4,5-c]pyridine,
2-(4-biphenylyl)-1-({(3S)-1-[(methyloxy)acetyl]-3-pyrrolidinyl}methyl)-1H-imidazo[4,5-c]pyridine,
(3S)-3-{[2-(4-biphenylyl)-1H-imidazo[4,5-c]pyridin-1-yl]methyl}-N,N-dimethyl-1-pyrrolidinecarboxamide,

(3S)-3-{[2-(4-biphenylyl)-1H-imidazo[4,5-c]pyridin-1-yl]methyl}-N-methyl-1-pyrrolidinecarboxamide,
2-(4-biphenylyl)-1-{[(3S)-1-(3,3,3-trifluoropropanoyl)-3-pyrrolidinyl]methyl}-1H-imidazo[4,5-c]pyridine,
2-[4-(1-benzofuran-5-yl)phenyl]-1-{[(3S)-1-(cyclopropylcarbonyl)-3-pyrrolidinyl]methyl}-1H-imidazo[4,5-c]pyridine,
1-{[(3S)-1-(cyclopropylcarbonyl)-3-pyrrolidinyl]methyl}-2-[4-(1H-indol-6-yl)phenyl]-1H-imidazo[4,5-c]pyridine,
1-{[(3S)-1-(cyclopropylcarbonyl)-3-pyrrolidinyl]methyl}-2-[4-(1H-indazol-6-yl)phenyl]-1H-imidazo[4,5-c]pyridine,
1-{[(3S)-1-(cyclopropylcarbonyl)-3-pyrrolidinyl]methyl}-2-[4-(1H-indol-5-yl)phenyl]-1H-imidazo[4,5-c]pyridine,
3-{[(3S)-1-(cyclopropylcarbonyl)-3-pyrrolidinyl]methyl}-2-(4′-fluoro-4-biphenylyl)-3H-imidazo[4,5-b]pyridine,
3-{[(3S)-1-(cyclopropylcarbonyl)-3-pyrrolidinyl]methyl}-2-[4′-(methyloxy)-4-biphenylyl]-3H-imidazo[4,5-b]pyridine,
3-{[(3S)-1-(cyclopropylcarbonyl)-3-pyrrolidinyl]methyl}-2-[4′-(ethyloxy)-4-biphenylyl]-3H-imidazo[4,5-b]pyridine,
3-{[(3S)-1-(cyclopropylcarbonyl)-3-pyrrolidinyl]methyl}-2-(2′,4′-dimethyl-4-biphenylyl)-3H-imidazo[4,5-b]pyridine,
4′-(3-{[(3S)-1-(cyclopropylcarbonyl)-3-pyrrolidinyl]methyl}-3H-imidazo[4,5-b]pyridin-2-yl)-3-biphenylcarboxylic acid,
2-(3′-chloro-4-biphenylyl)-3-{[(3S)-1-(cyclopropylcarbonyl)-3-pyrrolidinyl]methyl}-3H-imidazo[4,5-b]pyridine,
2-(4′-chloro-4-biphenylyl)-3-{[(3S)-1-(cyclopropylcarbonyl)-3-pyrrolidinyl]methyl}-3H-imidazo[4,5-b]pyridine,
2-(3′-chloro-4′-fluoro-4-biphenylyl)-3-{[(3S)-1-(cyclopropylcarbonyl)-3-pyrrolidinyl]methyl}-3H-imidazo[4,5-b]pyridine,
2-(4-biphenylyl)-3-{[(3S)-1-(cyclopropylcarbonyl)-3-pyrrolidinyl]methyl}-3H-imidazo[4,5-b]pyridine,
3-{[(3S)-1-(cyclopropylcarbonyl)-3-pyrrolidinyl]methyl}-2-[4-(1H-indol-6-yl)phenyl]-3H-imidazo[4,5-b]pyridine,
2-[4-(1-benzofuran-5-yl)phenyl]-3-{[(3S)-1-(cyclopropylcarbonyl)-3-pyrrolidinyl]methyl}-3H-imidazo[4,5-b]pyridine,
6-chloro-3-{[(3S)-1-(cyclopropylcarbonyl)-3-pyrrolidinyl]methyl}-2-(2′-methyl-4-biphenylyl)-3H-imidazo[4,5-b]pyridine,
6-chloro-3-{[(3S)-1-(cyclopropylcarbonyl)-3-pyrrolidinyl]methyl}-2-[3′-fluoro-4′-(methyloxy)-4-biphenylyl]-3H-imidazo[4,5-b]pyridine,
6-chloro-3-{[(3S)-1-(cyclopropylcarbonyl)-3-pyrrolidinyl]methyl}-2-(2′,4′-dimethyl-4-biphenylyl)-3H-imidazo[4,5-b]pyridine,
2-[4-(1-benzofuran-5-yl)phenyl]-6-chloro-3-{[(3S)-1-(cyclopropylcarbonyl)-3-pyrrolidinyl]methyl}-3H-imidazo[4,5-b]pyridine,
6-chloro-3-{[(3S)-1-(cyclopropylcarbonyl)-3-pyrrolidinyl]methyl}-2-[4-(1H-indazol-5-yl)phenyl]-3H-imidazo[4,5-b]pyridine,
2-[4-(1H-benzimidazol-5-yl)phenyl]-6-chloro-3-{[(3S)-1-(cyclopropylcarbonyl)-3-pyrrolidinyl]methyl}-3H-imidazo[4,5-b]pyridine,
6-chloro-3-{[(3S)-1-(cyclopropylcarbonyl)-3-pyrrolidinyl]methyl}-2-[4-(1H-indazol-6-yl)phenyl]-3H-imidazo[4,5-b]pyridine,
8-[4-(1-benzofuran-5-yl)phenyl]-6-chloro-9-{[(3S)-1-(cyclopropylcarbonyl)-3-pyrrolidinyl]methyl}-9H-purine,
6-chloro-9-{[(3S)-1-(cyclopropylcarbonyl)-3-pyrrolidinyl]methyl}-8-(2′,4′-dichloro-4-biphenylyl)-9H-purine,
8-(4-biphenylyl)-6-chloro-9-{[(3S)-1-(cyclopropylcarbonyl)-3-pyrrolidinyl]methyl}-9H-purine,
8-(4-biphenylyl)-9-{[(3S)-1-(cyclopropylcarbonyl)-3-pyrrolidinyl]methyl}-9H-purine,
8-[4-(1-benzofuran-5-yl)phenyl]-9-{[(3S)-1-(cyclopropylcarbonyl)-3-pyrrolidinyl]methyl}-9H-purine,
6-chloro-9-{[(3S)-1-(cyclopropylcarbonyl)-3-pyrrolidinyl]methyl}-8-(4′-fluoro-4-biphenylyl)-9H-purine,
9-{[(3S)-1-(cyclopropylcarbonyl)-3-pyrrolidinyl]methyl}-8-(4′-fluoro-4-biphenylyl)-6-(4-methyl-1-piperazinyl)-9H-purine,
1-{[(3S)-1-(cyclopropylcarbonyl)-3-pyrrolidinyl]methyl}-2-[4′-(methyloxy)-4-biphenylyl]-1H-imidazo[4,5-b]pyridine,
1-{[(3S)-1-(cyclopropylcarbonyl)-3-pyrrolidinyl]methyl}-2-[4-(1H-indol-5-yl)phenyl]-1H-imidazo[4,5-b]pyridine,
8-[4-(1-benzofuran-5-yl)phenyl]-9-{[(3S)-1-(cyclopropylcarbonyl)-3-pyrrolidinyl]methyl}-N-(1-methylethyl)-9H-purin-6-amine,
1-{[(3S)-1-(cyclopropylcarbonyl)-3-pyrrolidinyl]methyl}-2-[4-(1H-pyrrolo[2,3-b]pyridin-5-yl)phenyl]-1H-imidazo[4,5-c]pyridine,
1-{[(3S)-1-(cyclopropylcarbonyl)-3-pyrrolidinyl]methyl}-2-[4-(1H-indol-4-yl)phenyl]-1H-imidazo[4,5-c]pyridine,
1-{[(3S)-1-(cyclopropylcarbonyl)-3-pyrrolidinyl]methyl}-2-[4-(1H-indol-7-yl)phenyl]-1H-imidazo[4,5-c]pyridine,
1-{[(3S)-1-(cyclopropylcarbonyl)-3-pyrrolidinyl]methyl}-2-[4-(1H-pyrrolo[3,2-b]pyridin-6-yl)phenyl]-1H-imidazo[4,5-c]pyridine,
2-[4-(1,3-benzothiazol-5-yl)phenyl]-1-{[(3S)-1-(cyclopropylcarbonyl)-3-pyrrolidinyl]methyl}-1H-imidazo[4,5-c]pyridine,
1-{[(3S)-1-(cyclopropylcarbonyl)-3-pyrrolidinyl]methyl}-2-[4-(1H-indazol-4-yl)phenyl]-1H-imidazo[4,5-c]pyridine,
5-[4-(1-{[(3S)-1-(cyclopropylcarbonyl)-3-pyrrolidinyl]methyl}-1H-imidazo[4,5-c]pyridin-2-yl)phenyl]-1H-pyrazolo[3,4-b]pyridine,
2-[4-(1H-benzimidazol-5-yl)phenyl]-1-{[(3S)-1-(cyclopropylcarbonyl)-3-pyrrolidinyl]methyl}-1H-imidazo[4,5-c]pyridine,
1-{[(3S)-1-(cyclopropylcarbonyl)-3-pyrrolidinyl]methyl}-2-[4-(1H-pyrrolo[2,3-b]pyridin-4-yl)phenyl]-1H-imidazo[4,5-c]pyridine,
1-{[(3S)-1-(cyclopropylcarbonyl)-3-pyrrolidinyl]methyl}-2-(4-imidazo[1,2-a]pyridin-7-ylphenyl)-1H-imidazo[4,5-c]pyridine,
3-{[(3S)-1-(cyclopropylcarbonyl)-3-pyrrolidinyl]methyl}-2-[4-(1H-indol-6-yl)phenyl]-6-methyl-3H-imidazo[4,5-b]pyridine, and
3-{[(3S)-1-(cyclopropylcarbonyl)-3-pyrrolidinyl]methyl}-2-[4-(1H-indol-6-yl)phenyl]-5-(methyloxy)-3H-imidazo[4,5-b]pyridine.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthalene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound or salt represented by Formula (I) as mixtures with isomers thereof in which one or more chiral centers are inverted. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula (I), as well as any wholly or partially equilibrated mixtures thereof. The present invention also includes the individual isomers of the compound or salt represented by the Formula (I) as well as mixtures with isomers thereof in which one or more chiral centers are inverted. It is to be understood that the present invention includes all combinations and subsets of the particular groups defined hereinabove.

### DEFINITIONS

Terms are used within their accepted meanings. The following definitions are meant to clarify, but not limit, the terms defined.

As used herein, the term “alkyl” (or “alkylene”) refers to a straight or branched chain alkyl, preferably having from one to twelve carbon atoms, which may be unsubstituted or substituted, saturated or unsaturated with multiple degrees of substitution included within the present invention. Suitable substituents are selected from the group consisting of halogen, haloalkyl, cyclopropyl, alkoxy, acyl, amides, carboxylic acid, ester, cyano, hydroxyl, alkoxy, amino, substituted amino, alkylthio, alkylsulfonyl, and aminosulfonyl, oxazole and methylisoxazole. Examples of “alkyl” as used herein include methyl, ethyl, propyl, isopropyl, isobutyl, n-butyl, t-butyl, isopentyl, n-pentyl, and the like, as well as substituted versions thereof.

As used herein, the term “cycloalkyl” refers to an unsubstituted or substituted mono- or polycyclic non-aromatic saturated ring, which optionally includes an alkylene linker through which the cycloalkyl may be attached. Exemplary “cycloalkyl” groups include, but are not limited to, cyclopropyl, cyclobutyl, cyclopentyl, cyclohexyl, cycloheptyl, and the like, as well as unsubstituted and substituted versions thereof.

As used herein, the term “alkoxy” refers to the group —ORa, where Ra is C1-3alkyl or C3-7cycloalkyl as defined above.

As used herein, the term “substituted amino” is meant —NR′R″ wherein each R′ and R″ is independently selected from a group including hydrogen, C1-6alkyl, acyl, C3-C7cycloalkyl, wherein at least one of R′ and R″ is not hydrogen. Examples of substituted amino includes, but are not limited to alkylamino, dialkylamino, acylamino, and cycloalkylamino.

As used herein, the term “heterocycle” or “heterocyclyl” or “heterocycloalkyl” refers to unsubstituted and substituted mono- or polycyclic non-aromatic ring system containing one or more heteroatoms. Preferred heteroatoms include N, O, and S, including N-oxides, sulfur oxides, and dioxides. Preferably the ring is three to eight-membered and is either fully saturated or has one or more degrees of unsaturation. Multiple degrees of substitution are included within the present definition. Examples of “heterocyclic” groups include, but are not limited to tetrahydrofuranyl, pyranyl, 1,4-dioxanyl, 1,3-dioxanyl, piperidinyl, pyrrolidinyl, morpholinyl, azetidinyl, piperazinyl, pyrrolidinonyl, piperazinonyl, pyrazolidinyl, and their various tautomers, as well as unsubstituted and substituted versions thereof.

As used herein, the term “aryl”, unless otherwise defined, is meant aromatic, hydrocarbon, ring system. The ring system may be monocyclic or fused polycyclic (e.g., bicyclic, tricyclic, etc.), substituted or unsubstituted. In various embodiments, the monocyclic aryl ring is C5-C10, or C5-C7, or C5-C6, where these carbon numbers refer to the number of carbon atoms that form the ring system. A C6 ring system, i.e. a phenyl ring, is a suitable aryl group. In various embodiments, the polycyclic ring is a bicyclic aryl group, where suitable bicyclic aryl groups are C8-C12, or C9-C10. A naphthyl ring, which has 10 carbon atoms, is a suitable polycyclic aryl group. Suitable substituents for aryl are described in the definition of “optionally substituted”.

As used herein, the term “heteroaryl”, unless otherwise defined, is meant an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 8 hetero atoms. A polycyclic heteroaryl ring may contain fused, spiro or bridged ring junctions, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Exemplary monocyclic heteroaryl include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl. Exemplary heteroaryl groups include, but are not limited to benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Suitable substituents for heteroaryl are described in the definition of “optionally substituted”.

As used herein, the term “cyano” refers to the group —CN.

As used herein, the term “acyl” refers to the group —C(O)Rb, where Rb is alkyl, cycloalkyl, or heterocyclyl, as each is defined herein.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, with one or more substitutent group. The phrase should not be interpreted as duplicative of the substitutions herein described and depicted. Exemplary optional substituent groups include acyl, C₁₋₆alkyl, alkylsulfonyl, alkoxy, cyano, carboxylic acid, ester, halogen, haloalkyl, hydroxyl, oxo, amide, amino, substituted amino, alkylthio, sulfonamide, sulfamide, urea, thiourea and nitro.

The invention further provides a pharmaceutical composition (also referred to as pharmaceutical formulation) comprising a compound of Formula (I) or pharmaceutically acceptable salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

In accordance with another aspect of the invention there is provided a process for the preparation of a pharmaceutical composition comprising mixing (or admixing) a compound of Formula (I) or salt thereof with at least one excipient.

### Pharmaceutical Compositions

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

The present invention provides a method of treatment in a mammal, especially a human, suffering from disease conditions targeted by the present compounds. Such treatment comprises the step of administering a therapeutically effective amount of a compound of Formula (I) or salt thereof to said mammal, particularly a human. Treatment can also comprise the step of administering a therapeutically effective amount of a pharmaceutical composition containing a compound of Formula (I) or salt thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

While it is possible that, for use in therapy, a therapeutically effective amount of a compound of Formula (I) or salt thereof may be administered as the raw chemical, it is typically presented as the active ingredient of a pharmaceutical composition or formulation.

The precise therapeutically effective amount of a compound or salt thereof of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula (I) or salt thereof will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt thereof may be determined as a proportion of the effective amount of the compound of Formula (I) per se. Similar dosages should be appropriate for treatment (including prophylaxis) of the other conditions referred herein for treatment. In general, determination of appropriate dosing can be readily arrived at by one skilled in medicine or the pharmacy art.

### Combinations

When a compound of Formula (I) is administered for the treatment of cancer, the term “co-administering” and derivatives thereof as used herein is meant either simultaneous administration or any manner of separate sequential administration of a FAS inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present FAS inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine[1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloro ethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINEC®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2(1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I—DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I—DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

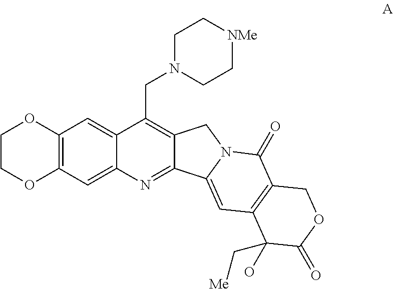

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2Activity by a monoclonal Anti-VEGF antibody blocks tumor growth in mice, Cancer Res. (2000) 60, 5117-5124).

Non-receptor kinase angiogenesis inhibitors may also find use in the present invention Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of Formula (I) and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

### EXPERIMENTALS

### Preparation

The derivatives described herein were prepared by the general methods described below. Formulas and R group designations used in the schemes below are meant to be used for this section only, and they may be inconsistent with those in the claims.

The pyrrolidine amide or carbamate intermediates (A1, A2) can be prepared according to Scheme I from either racemic or optically-active starting material.

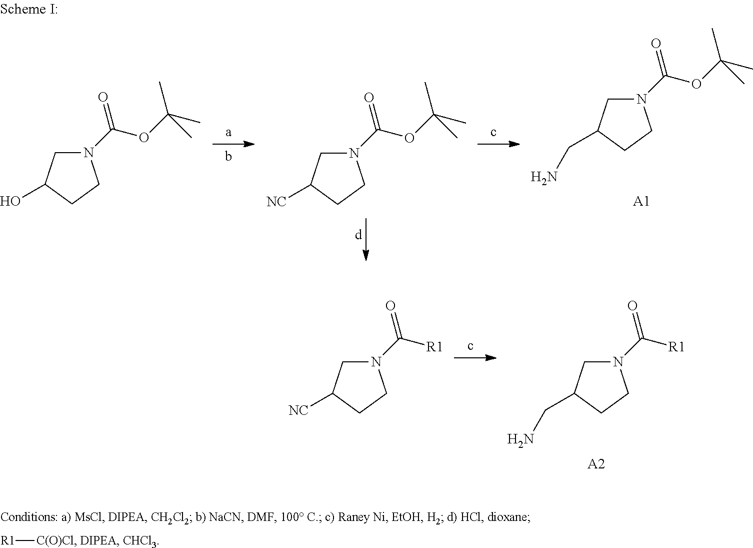

The biaryl aldehyde intermediate B3 can be prepared through Suzuki coupling of the two halves, as outlined in Scheme II.

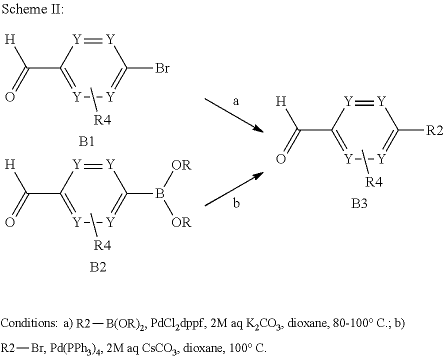

Displacement of the heteroaryl halide with pyrrolidine A1 or A2 can usually be accomplished with heat in a polar solvent as shown in Scheme III. Alternative methods for coupling the two pieces together may include metal-catalyzed C—N bond-forming reactions.

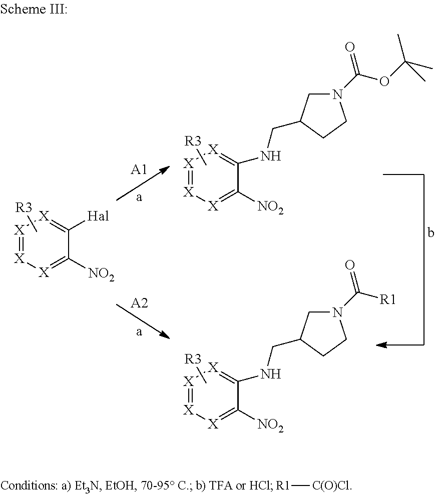

Formation of the azabenzimidazole can proceed by reduction of the nitro group and condensation of the aniline with the biaryl aldehyde B3 (Scheme IV).

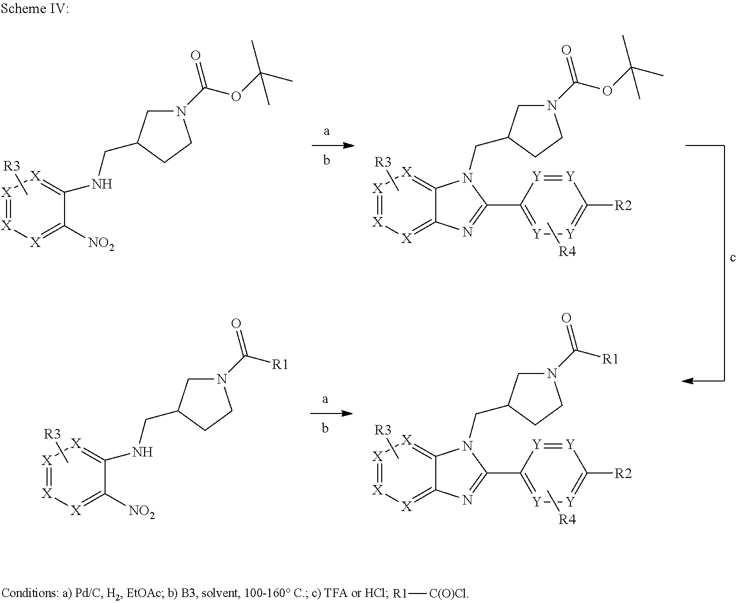

The azabenzimidazoles can also be prepared in a more stepwise manner through reduction of the nitro group, condensation with a bromo-aryl-aldehyde, and Suzuki coupling with a boronic acid, as seen in Scheme V.

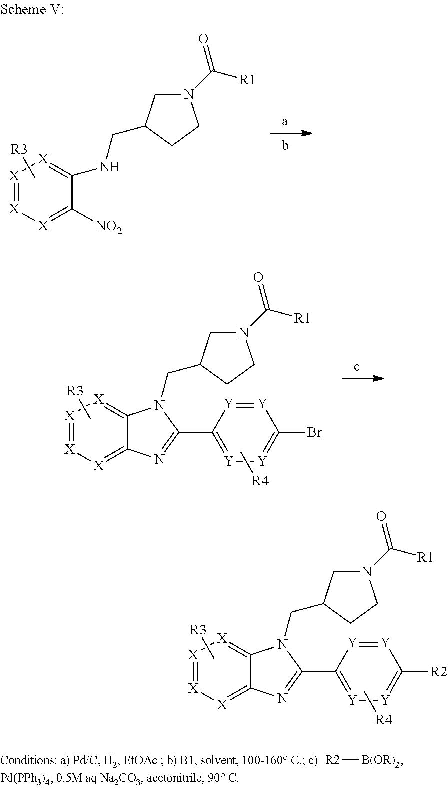

### Intermediate 1

### 1,1-dimethylethyl(3S)-3-(aminomethyl)-1-pyrrolidinecarboxylate

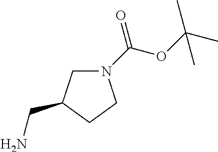

### a) 1,1-dimethylethyl(3S)-3-[(methylsulfonyl)oxy]-1-pyrrolidinecarboxylate

A solution of 1,1-dimethylethyl(3S)-3-hydroxy-1-pyrrolidinecarboxylate (166 mmol) and N,N-diisopropylethylamine (265 mmol) in dichloromethane (200 mL) at 0° C. under nitrogen atmosphere was treated with methanesulfonyl chloride (199 mmol) in dichloromethane and allowed to warm to ambient over 1 h. Analysis by LCMS indicated the reaction was complete. The mixture was washed with 1M aq hydrochloric acid and brine, dried over sodium sulfate, filtered, and concentrated in vacuo. Purification of the residue by flash chromatography (0-5% methanol/dichloromethane) gave the title product in quantitative yield (166 mmol). ¹H NMR (400 MHz, CDCl₃) δ ppm 1.49 (s, 9H) 2.08-2.21 (m, 1H) 2.29 (br. s., 1H) 3.07 (s, 3H) 3.36-3.64 (m, 3H) 3.65-3.75 (m, 1H) 5.28 (tt, J=4.23, 2.08 Hz, 1H).

### b) 1,1-dimethylethyl(3R)-3-cyano-1-pyrrolidinecarboxylate

A mixture of 1,1-dimethylethyl(3S)-3-[(methylsulfonyl)oxy]-1-pyrrolidinecarboxylate (211 mmol) and sodium cyanide (633 mmol) in N,N-dimethylformamide (300 mL) was vigorously stirred with a mechanical stirrer while heating at 100° C. under a nitrogen atmosphere for 18 h. The mixture was allowed to cool to ambient temperature, filtered, and washed thoroughly with diethyl ether. The filtrate was diluted with dilute brine and extracted with diethyl ether (4×700 mL). The combined organic extracts were washed with dilute brine, filtered through a pad of sodium sulfate, and concentrated in vacuo. Purification of the residue by flash chromatography (0-50% ethyl acetate/hexanes) gave the title product (141 mmol, 67% yield). ¹H NMR (400 MHz, CDCl₃) δ ppm 1.48 (s, 9H) 2.14-2.37 (m, 2H) 3.00-3.20 (m, 1H) 3.45 (dt, J=11.05, 6.98 Hz, 1H) 3.53-3.66 (m, 2H) 3.65-3.76 (m, 1H).

### c) 1,1-dimethylethyl(3S)-3-(aminomethyl)-1-pyrrolidinecarboxylate

A solution of 1,1-dimethylethyl(3R)-3-cyano-1-pyrrolidinecarboxylate (73.9 mmol) in ethanol (100 mL) was added to Raney Nickel (73.9 mmol; 2 scoops of Raney Nickel in water) in a Parr bottle under a stream of nitrogen. The mixture was well flushed with nitrogen then placed on a Parr shaker under hydrogen atmosphere at 60 psi for overnight. The mixture (under N₂ stream) was filtered through Celite, washed with a little ethanol, and then the filter cake was immediately doused with water. The ethanol solution was concentrated in vacuo to afford the title product as a clear oil (71.9 mmol, 97% yield). The product was determined to be a 95:5 ratio of enantiomers (i.e., 90% ee). The use of chiral HPLC now (Daicel Chiralpak AD-H column (4.6×150 mm) with a mobile phase of heptane:ethanol:isopropylamine (85:10:0.1), a flow rate of 1.0 mL/min, and UV detection at 215 nm gave a retention time of 4.4 min for the 1,1-dimethylethyl(3R)-3-(aminomethyl)-1-pyrrolidinecarboxylate and 4.8 min for the 1,1-dimethylethyl(3S)-3-(aminomethyl)-1-pyrrolidinecarboxylate) or with a more advanced intermediate afforded the product in >99% ee. ¹H NMR (400 MHz, CDCl₃) δ ppm 1.47 (s, 9H) 1.54-1.73 (m, 1H) 1.91-2.13 (m, 1H) 2.28-2.52 (m, 1H) 2.78-2.93 (m, 1H) 2.95-3.16 (m, 1H) 3.24-3.67 (m, 5H).

### Intermediate 2

### {[(3S)-1-(cyclopropylcarbonyl)-3-pyrrolidinyl]methyl}amine

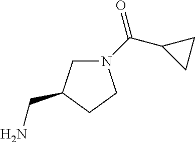

### a) (3R)-1-(cyclopropylcarbonyl)-3-pyrrolidinecarbonitrile

A solution of 1,1-dimethylethyl(3R)-3-cyano-1-pyrrolidinecarboxylate (138 mmol) in ethanol (200 mL) was treated with 4N HCl in dioxane (480 mmol) and stirred for 2 h. The mixture was concentrated in vacuo to an oil and then azeotroped with ethanol and chloroform. The residue was dissolved in chloroform (300 mL) and treated with N,N-diisopropylethylamine (413 mmol) and cooled over an ice bath. The mixture was treated with cyclopropanecarbonyl chloride (165 mmol) in chloroform (100 mL) and then the ice bath was removed and the mixture stirred for 2 h. The mixture was washed with 1N hydrochloric acid and brine, dried over sodium sulfate, filtered, and concentrated in vacuo. Purification of the residue by flash chromatography (0-5% methanol/dichloromethane) gave the title product (134 mmol, 97% yield). ¹H NMR (400 MHz, CDCl₃) δ ppm 0.73-0.91 (m, 2H) 0.96-1.10 (m, 2H) 1.47-1.81 (m, 1H) 2.08-2.52 (m, 2H) 3.03-3.33 (m, 1H) 3.48-4.13 (m, 4H).

### b) {[(3S)-1-(cyclopropylcarbonyl)-3-pyrrolidinyl]methyl}amine

A solution of (3R)-1-(cyclopropylcarbonyl)-3-pyrrolidinecarbonitrile (28.3 mmol) in ethanol (300 mL) and ammonia solution (15 mL, 28.3 mmol) was flushed with nitrogen and raney nickel catalyst (1 scoop) was added. The mixture was placed on a Parr shaker and flushed several times with nitrogen and then shaken under a hydrogen atmosphere at 60 psi for 3 h. The mixture was flushed with nitrogen and filtered through Celite under a nitrogen atmosphere (keeping the catalyst wet), and then the filter cake was washed with a little ethanol then immediately doused with water.

The solvent was evaporated to provide the product as a clear oil (25.6 mmol, 90% yield). Analysis by chiral HPLC indicated 75% of the title product, 2.4% of the other enantiomer, and ˜12% of the bis{[(3S)-1-(cyclopropylcarbonyl)-3-pyrrolidinyl]methyl}amine by-product. A sample of {[(3S)-1-(cyclopropylcarbonyl)-3-pyrrolidinyl]methyl}amine (1.06 g) was purified by chiral HPLC (Chiralpak AD 20μ column (101×250 mm) with a mobile phase of heptane:ethanol:isopropylamine (75:25:0.1), a flow rate of 500 mL/min, and UV detection at 220 nm gave a retention time of 8.5 min for the {[(3R)-1-(cyclopropylcarbonyl)-3-pyrrolidinyl]methyl}amine and 11 min for the {[(3S)-1-(cyclopropylcarbonyl)-3-pyrrolidinyl]methyl}amine) to yield 720 mg (68% recovery) of chirally (>99% ee) and chemically pure title compound. ¹H NMR (400 MHz, CDCl₃) δ ppm 0.69-0.83 (m, 2H) 0.91-1.09 (m, 2H) 1.28 (br. s., 2H) 1.52-1.82 (m, 2H) 1.97-2.20 (m, 1H) 2.20-2.45 (m, 1H) 2.67-2.98 (m, 2H) 3.06-3.38 (m, 1H) 3.38-3.92 (m, 3H).

### Example 1

### 4′-(1-{[(3S)-1-(cyclopropylcarbonyl)-3-pyrrolidinyl]methyl}-1H-imidazo[4,5-b]pyridin-2-yl)-3-biphenylol

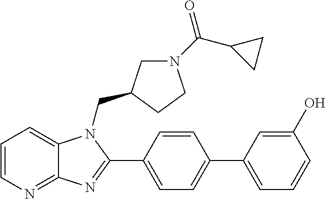

a) A solution of N³-{[(3S)-1-(cyclopropylcarbonyl)-3-pyrrolidinyl]methyl}-2,3-pyridinediamine (0.31 mmol) and 3′-hydroxy-4-biphenylcarbaldehyde (0.31 mmol) in N-methyl-2-pyrrolidone (3 mL) was heated at 140° C. for 30 h. The reaction mixture was poured into water (15 mL) and stirred at room temperature. The mixture was extracted with ethyl acetate (30 mL) and the organic layer was washed with water (3×10 mL) and brine (2×10 mL), dried over sodium sulfate, filtered, and concentrated in vacuo. The residue was purified by reverse phase HPLC to provide the title product as a solid (36%). MS(ES)+ m/e 439.2 [M+H]⁺.

### Example 2

### 1-{[(3S)-1-(cyclopropylcarbonyl)-3-pyrrolidinyl]methyl}-2-(3′-methyl-4-biphenylyl)-1H-imidazo[4,5-b]pyridine

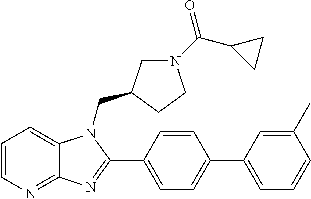

a) Following the procedure of example 1a using 3′-methyl-4-biphenylcarbaldehyde afforded the title product as a solid (47%). MS(ES)+ m/e 437.2 [M+H]⁺.

### Example 3

### 1-{[(3S)-1-(cyclopropylcarbonyl)-3-pyrrolidinyl]methyl}-2-(2′,4′-dimethyl-4-biphenylyl)-1H-imidazo[4,5-b]pyridine

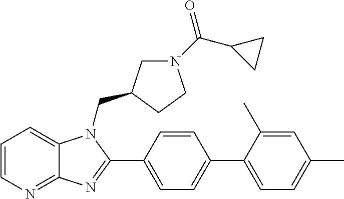

a) Following the procedure of example 1a using 2′,4′-dimethyl-4-biphenylcarbaldehyde afforded the title product as a solid (24%). MS(ES)+ m/e 451.2 [M+H]⁺.

### Example 4

### 2-[2′-chloro-4′-(methyloxy)-4-biphenylyl]-1-{[(3S)-1-(cyclopropylcarbonyl)-3-pyrrolidinyl]methyl}-1H-imidazo[4,5-b]pyridine

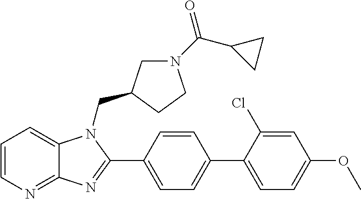

### a) 2′-chloro-4′-(methyloxy)-4-biphenylcarbaldehyde

Following the procedure described in example 29a using [2-chloro-4-(methyloxy)phenyl]boronic acid and purification by flash chromatography gave the title product as a solid (75%).

### b) 2-[2′-chloro-4′-(methyloxy)-4-biphenylyl]-1-{[(3S)-1-(cyclopropylcarbonyl)-3-pyrrolidinyl]methyl}-1H-imidazo[4,5-b]pyridine

Following the procedure of example 1a using 2′-chloro-4′-(methyloxy)-4-biphenylcarbaldehyde afforded the title product as a solid (50%). MS(ES)+ m/e 487.2 [M+H]⁺.

### Example 5

### 1-{[(3S)-1-(cyclopropylcarbonyl)-3-pyrrolidinyl]methyl}-2-[4-(1H-indazol-5-yl)phenyl]-1H-imidazo[4,5-b]pyridine

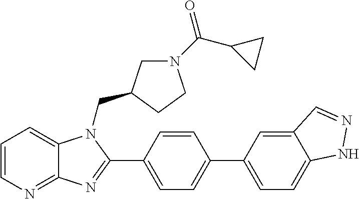

### a) 4-(1H-indazol-5-yl)benzaldehyde

Following the procedure described in examples 43a/b using 5-bromo-1H-indazole gave the title product as a solid (38%).

### b) 1-{[(3S)-1-(cyclopropylcarbonyl)-3-pyrrolidinyl]methyl}-2-[4-(1H-indazol-5-yl)phenyl]-1H-imidazo[4,5-b]pyridine

Following the procedure of example 1a using 4-(1H-indazol-5-yl)benzaldehyde afforded the title product as a solid (13%). MS(ES)+ m/e 463.2 [M+H]⁺.

### Example 6

### 1-{[(3S)-1-(cyclopropylcarbonyl)-3-pyrrolidinyl]methyl}-2-[4-(1H-indol-6-yl)phenyl]-1H-imidazo[4,5-b]pyridine

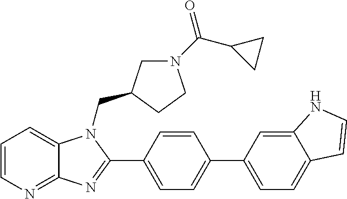

### a) 4-(1H-indol-6-yl)benzaldehyde

Following the procedure described in example 36a using 6-bromo-1H-indole and purification by reverse phase HPLC gave the title product as a solid (75%).

### b) 1-{[(3S)-1-(cyclopropylcarbonyl)-3-pyrrolidinyl]methyl}-2-[4-(1H-indol-6-yl)phenyl]-1H-imidazo[4,5-b]pyridine

Following the procedure of example 1a using 4-(1H-indol-6-yl)benzaldehyde afforded the title product as a solid (10%). MS(ES)+ m/e 462.2 [M+H]⁺.

### Example 7

### 1-{[(3S)-1-(cyclopropylcarbonyl)-3-pyrrolidinyl]methyl}-2-(4′-methyl-4-biphenylyl)-1H-imidazo[4,5-b]pyridine

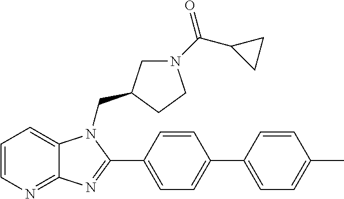

a) Following the procedure of example 1a using 4′-methyl-4-biphenylcarbaldehyde afforded the title product as a solid (31%). MS(ES)+ m/e 437.2 [M+H]⁺.

### Example 8

### 1-{[(3S)-1-(cyclopropylcarbonyl)-3-pyrrolidinyl]methyl}-2-(2′,4′-dichloro-4-biphenylyl)-1H-imidazo[4,5-b]pyridine

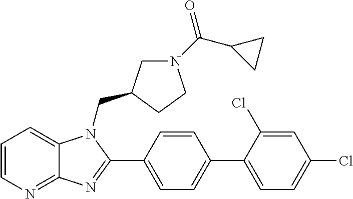

### a) 2′,4′-dichloro-4-biphenylcarbaldehyde

Following the procedure described in example 29a using (2,4-dichlorophenyl)boronic acid gave the title product as a solid (15%).

### b) 1-{[(3S)-1-(cyclopropylcarbonyl)-3-pyrrolidinyl]methyl}-2-(2′,4′-dichloro-4-biphenylyl)-1H-imidazo[4,5-b]pyridine

Following the procedure of example 1a using 2′,4′-dichloro-4-biphenylcarbaldehyde afforded the title product as a solid (39%). MS(ES)+ m/e 491.1 [M+H]⁺.

### Example 9

### 1-{[(3S)-1-(cyclopropylcarbonyl)-3-pyrrolidinyl]methyl}-2-(4′-fluoro-3′-methyl-4-biphenylyl)-1H-imidazo[4,5-b]pyridine

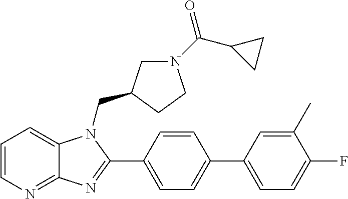

### a) 4′-fluoro-3′-methyl-4-biphenylcarbaldehyde

Following the procedure described in example 29a using (4-fluoro-3-methylphenyl)boronic acid gave the title product as a solid (65%).

### b) 1-{[(3S)-1-(cyclopropylcarbonyl)-3-pyrrolidinyl]methyl}-2-(4′-fluoro-3′-methyl-4-biphenylyl)-1H-imidazo[4,5-b]pyridine

Following the procedure of example 1a using 4′-fluoro-3′-methyl-4-biphenylcarbaldehyde afforded the title product as a solid (31%). MS(ES)+ m/e 455.2 [M+H]⁺.

### Example 10

### 2-(4-biphenylyl)-1-{[(3S)-1-(cyclopropylcarbonyl)-3-pyrrolidinyl]methyl}-1H-imidazo[4,5-b]pyridine

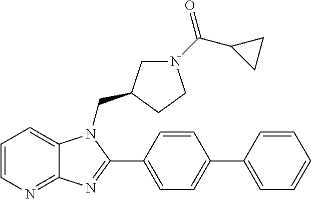

a) Following the procedure of example 1a using 4-biphenylcarbaldehyde afforded the title product as a solid (51%). MS(ES)+ m/e 423.2 [M+H]⁺.

### Example 11

### 2-[4-(1-benzofuran-5-yl)phenyl]-1-{[(3S)-1-(cyclopropylcarbonyl)-3-pyrrolidinyl]methyl}-1H-imidazo[4,5-b]pyridine

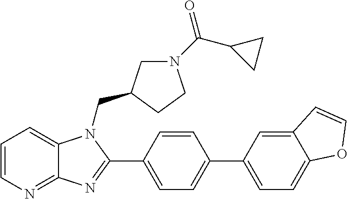

a) Following the procedure of example 1a using 4-(1-benzofuran-5-yl)benzaldehyde afforded the title product as a solid (23%). MS(ES)+ m/e 463.2 [M+H]⁺.

### Example 12

### 1-{[(3S)-1-(cyclopropylcarbonyl)-3-pyrrolidinyl]methyl}-2-(4′-fluoro-4-biphenylyl)-1H-imidazo[4,5-b]pyridine

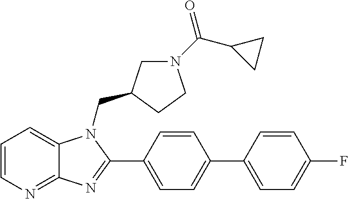

a) Following the procedure of example 1a using 4′-fluoro-4-biphenylcarbaldehyde afforded the title product as a solid (33%). MS(ES)+ m/e 441.2 [M+H]⁺.

### Example 13

### 2-(4-biphenylyl)-1-{[1-(cyclopropylcarbonyl)-3-pyrrolidinyl]methyl}-1H-imidazo[4,5-c]pyridine

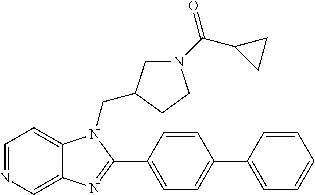

### a) 1,1-dimethylethyl 3-{[(3-nitro-4-pyridinyl)amino]methyl}-1-pyrrolidinecarboxylate

A solution of 4-chloro-3-nitropyridine (3.41 mmol) and 1,1-dimethylethyl 3-(aminomethyl)-1-pyrrolidinecarboxylate (3.58 mmol) in isopropanol (10 mL) was heated at 35° C. overnight. The reaction mixture was then concentrated in vacuo and the residue was purified by flash chromatography (50-100% ethyl acetate/hexanes) to afford the title product as a yellow resin (156 mg). MS(ES)+ m/e 323 [M+H]⁺.

### b) 1,1-dimethylethyl 3-{[2-(4-biphenylyl)-1H-imidazo[4,5-c]pyridin-1-yl]methyl}-1-pyrrolidinecarboxylate

A mixture of 1,1-dimethylethyl 3-{[(3-nitro-4-pyridinyl)amino]methyl}-1-pyrrolidinecarboxylate (0.155 mmol), 4-biphenylcarbaldehyde (0.155 mmol), and sodium dithionite (0.465 mmol) in water (1 mL) and ethanol (2 mL) was heated at 70° C. for 5 days. The reaction mixture was concentrated in vacuo and partitioned between 2M aq ammonium hydroxide (2 mL) and ethyl acetate (15 mL). The layers were separated, and the aqueous layer was extracted with (2×15 mL) ethyl acetate. The combined organic layers were dried over sodium sulfate, filtered, and were concentrated in vacuo. Purification of the residue by reverse phase HPLC and concentration of the product fractions provided 23 mg of a white solid. MS(ES)+ m/e 455 [M+H]⁺.

### c) 2-(4-biphenylyl)-1-{[1-(cyclopropylcarbonyl)-3-pyrrolidinyl]methyl}-1H-imidazo[4,5-c]pyridine

A solution of 1,1-dimethylethyl 3-{[2-(4-biphenylyl)-1H-imidazo[4,5-c]pyridin-1-yl]methyl}-1-pyrrolidinecarboxylate (0.051 mmol) in dichloromethane (2 mL) was treated dropwise with trifluoroacetic acid (˜0.5 mL) and stirred at room temperature until the starting material was consumed as detected by analytical HPLC. The reaction was concentrated in vacuo and then concentrated from dichloromethane twice to give a colorless resin (25 mg). The solid was diluted with dichloromethane (2 mL), cooled to 0° C., and treated with diisopropylethylamine (0.126 mmol) and cyclopropylcarbonyl chloride (0.053 mmol). The reaction was warmed to room temperature, diluted with dichloromethane, and washed with saturated aq sodium bicarbonate. The organic layer was dried over sodium sulfate, filtered, and concentrated in vacuo. Purification of the residue by reverse phase HPLC provided the title product as a white trifluoroacetate salt (18 mg). MS(ES)+ m/e 423 [M+H]⁺.

### Example 14

### 1-{[1-(cyclopropylcarbonyl)-3-pyrrolidinyl]methyl}-2-[4-(1H-indazol-5-yl)phenyl]-1H-imidazo[4,5-c]pyridine

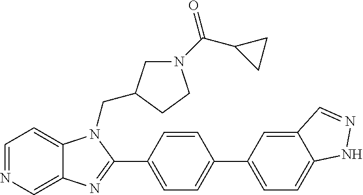

### a) 4-(1H-indazol-5-yl)benzaldehyde

A mixture of 5-bromo-1H-indazole (1.02 mmol), 4-formylphenylboronic acid (1.32 mmol), tetrakis(triphenylphosphine)palladium(0) (0.051 mmol) and sodium bicarbonate (3.05 mmol) in water (3 mL) and N,N-dimethylformamide (3 mL) was irradiated in the microwave at 150° C. for 30 min. The reaction mixture was diluted with water and extracted with (3×20 mL) ethyl acetate. The combined organic layers were dried over sodium sulfate, filtered, and concentrated in vacuo. Purification of the residue by flash chromatography (5-50% ethyl acetate/hexanes) gave the title product as a pale yellow solid (34%). MS(ES)+ m/e 223 [M+H]⁺.

### b) N-{[1-(cyclopropylcarbonyl)-3-pyrrolidinyl]methyl}-3-nitro-4-pyridinamine

A solution of 1,1-dimethylethyl 3-{[(3-nitro-4-pyridinyl)amino]methyl}-1-pyrrolidinecarboxylate (0.776 mmol) in dichloromethane (10 mL) was treated dropwise with trifluoroacetic acid (0.6 mL) and stirred at room temperature until the starting material was consumed as detected by analytical HPLC. The reaction was concentrated in vacuo and then concentrated from dichloromethane twice, methanol twice, and dichloromethane once. The residue was diluted with ethyl acetate, passed through a short plug of basic alumina, and concentrated in vacuo. The residue was diluted with dichloromethane (10 mL), cooled to 0° C., and treated with diisopropylethylamine (1.94 mmol) and cyclopropylcarbonyl chloride (0.814 mmol). After 1 h, the reaction was warmed to room temperature, treated with a few drops of methanol, diluted with dichloromethane, and washed with saturated aq sodium bicarbonate. The organic layer was dried over sodium sulfate, filtered, and concentrated in vacuo to provide the title product as a yellow solid (200 mg). MS(ES)+ m/e 291 [M+H]⁺.

### c) N⁴-{[1-(cyclopropylcarbonyl)-3-pyrrolidinyl]methyl}-3,4-pyridinediamine

A solution of N-{[1-(cyclopropylcarbonyl)-3-pyrrolidinyl]methyl}-3-nitro-4-pyridinamine (0.689 mmol) in ethanol (5 mL) was treated with 10% palladium on carbon (4 mg) and reduced under an atmosphere of hydrogen. After 2 h, the reaction mixture was evacuated and backfilled with nitrogen. Starting material remained, so additional catalyst was added (4 mg) and the reaction mixture was reduced under hydrogen overnight. After evacuating the reaction mixture and confirming the reaction had gone to completion, the mixture was filtered through Celite and concentrated in vacuo to afford the crude title compound as a light yellow resin (quantitative). MS(ES)+ m/e 261 [M+H]⁺.

### d) 1-{[1-(cyclopropylcarbonyl)-3-pyrrolidinyl]methyl}-2-[4-(1H-indazol-5-yl)phenyl]-1H-imidazo[4,5-c]pyridine

A solution of crude N⁴-{[1-(cyclopropylcarbonyl)-3-pyrrolidinyl]methyl}-3,4-pyridinediamine (0.157 mmol) and 4-(1H-indazol-5-yl)benzaldehyde (0.157 mmol) in n-butanol (5 mL) was heated at 85° C. for 3 days. The reaction mixture was concentrated in vacuo and the residue was purified by reverse phase HPLC. Product fractions were combined, neutralized with saturated aq sodium bicarbonate, extracted with dichloromethane, dried over sodium sulfate, and concentrated in vacuo to afford the title product as an amorphous yellow solid (76%). MS(ES)+ m/e 463 [M+H]⁺.

### Example 15

### 1-{[(3S)-1-acetyl-3-pyrrolidinyl]methyl}-2-(4-biphenylyl)-1H-imidazo[4,5-c]pyridine

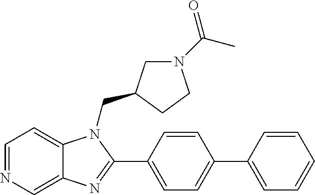

### a) 1,1-dimethylethyl(3S)-3-{[(3-amino-4-pyridinyl)amino]methyl}-1-pyrrolidinecarboxylate

A solution of 1,1-dimethylethyl(3S)-3-{[(3-nitro-4-pyridinyl)amino]methyl}-1-pyrrolidinecarboxylate (4.9 mmol) in ethanol (10 mL) was treated with 10% palladium on carbon (30 mg) and reduced under an atmosphere of hydrogen. After 2 h, the reaction mixture was evacuated and backfilled with nitrogen. Starting material remained, so additional catalyst was added (30 mg) and the reaction mixture was reduced under hydrogen overnight. After evacuating the reaction mixture and confirming the reaction had gone to completion, the mixture was filtered through Celite and concentrated in vacuo to afford the crude title compound as a light yellow resin (quantitative). MS(ES)+m/e 293 [M+H]⁺.

### b) 1,1-dimethylethyl(3S)-3-{[2-(4-biphenylyl)-1H-imidazo[4,5-c]pyridin-1-yl]methyl}-1-pyrrolidinecarboxylate

A solution of 1,1-dimethylethyl(3S)-3-{[(3-amino-4-pyridinyl)amino]methyl}-1-pyrrolidinecarboxylate (2.05 mmol) and 4-biphenylcarbaldehyde (2.05 mmol) in n-butanol (5 mL) was heated at 85° C. for 3 days. The reaction mixture was then treated with 10% palladium on carbon at 85° C. for 3 h to oxidize the dihydroimidazole intermediate. The reaction mixture was then filtered through Celite, washed with ethanol, and concentrated in vacuo. Purification of the crude residue by flash chromatography (100% ethyl acetate, then 1% Et₃N/5% EtOH/ethyl acetate) gave the title product as a yellow amorphous solid (0.748 g). MS(ES)+ m/e 455 [M+H]⁺.

### c) 2-(4-biphenylyl)-1-[(3R)-3-pyrrolidinylmethyl]-1H-imidazo[4,5-c]pyridine

A solution of 1,1-dimethylethyl(3S)-3-{[2-(4-biphenylyl)-1H-imidazo[4,5-c]pyridin-1-yl]methyl}-1-pyrrolidinecarboxylate (1.10 mmol) in THF (5 mL) was treated with 4M HCl in dioxane (1.7 mL) and stirred at room temperature overnight. Analysis by HPLC indicated remaining starting material, so more 4M HCl in dioxane (1.7 mL) was added and the reaction mixture was concentrated in vacuo, completing removal of the protecting group. The residue was triturated with ether and concentrated in vacuo until an amorphous hydrochloride salt was obtained (0.527 g). The sample was then partitioned between saturated aq sodium bicarbonate and dichloromethane, and the aqueous layer was extracted with dichloromethane until no chromophoric material remained in the aqueous layer. The combined organic layers were dried over sodium sulfate, filtered, and concentrated in vacuo to provide the title product as a yellow foam (0.390 g). MS(ES)+ m/e 355 [M+H]⁺.

### d) 1-{[(3S)-1-acetyl-3-pyrrolidinyl]methyl}-2-(4-biphenylyl)-1H-imidazo[4,5-c]pyridine

A solution of 2-(4-biphenylyl)-1-[(3R)-3-pyrrolidinylmethyl]-1H-imidazo[4,5-c]pyridine (0.11 mmol) and diisopropylethylamine (0.16 mmol) in dichloromethane (2 mL) was treated with acetyl chloride (0.12 mmol) and stirred at room temperature. After 2 h, the reaction mixture was diluted with dichloromethane and washed with saturated aq sodium bicarbonate. The organic layer was dried over sodium sulfate, filtered, and concentrated in vacuo. Purification of the residue by reverse phase HPLC, neutralization of the product fractions through partitioning between saturated aq sodium bicarbonate and dichloromethane, and concentration of the dried organic layers afforded the title product as an amorphous solid (42 mg). MS(ES)+ m/e 397 [M+H]⁺.

### Example 16

### 2-(4-biphenylyl)-1-{[(3S)-1-propanoyl-3-pyrrolidinyl]methyl}-1H-imidazo[4,5-c]pyridine

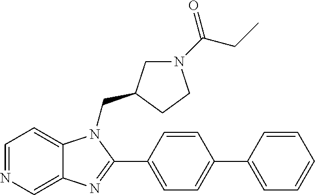

a) Following the procedure described in example 15d with propanoyl chloride provided the title compound as an off-white solid (44 mg). MS(ES)+ m/e 411 [M+H]⁺.

### Example 17

### 2-(4-biphenylyl)-1-{[(3S)-1-butanoyl-3-pyrrolidinyl]methyl}-1H-imidazo[4,5-c]pyridine

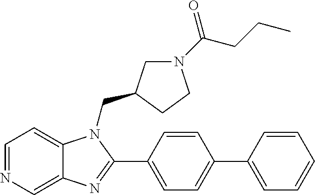

a) Following the procedure described in example 15d with butanoyl chloride provided the title compound as an off-white solid (46 mg). MS(ES)+ m/e 425 [M+H]⁺.

### Example 18

### 2-(4-biphenylyl)-1-({(3S)-1-[(methyloxy)acetyl]-3-pyrrolidinyl}methyl)-1H-imidazo[4,5-c]pyridine

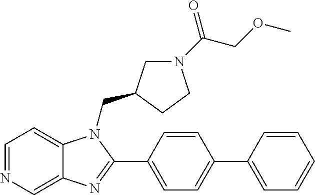

a) Following the procedure described in example 15d with methoxyacetyl chloride provided the title compound as an off-white solid (46 mg). MS(ES)+ m/e 427 [M+H]⁺.

### Example 19

### (3S)-3-{[2-(4-biphenylyl)-1H-imidazo[4,5-c]pyridin-1-yl]methyl}-N,N-dimethyl-1-pyrrolidinecarboxamide

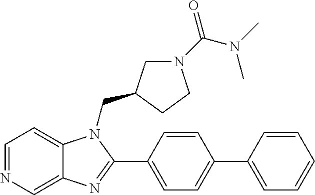

a) Following the procedure described in example 15d with N,N-dimethylcarbamyl chloride provided the title compound as an orange solid (58 mg). MS(ES)+ m/e 426 [M+H]⁺.

### Example 20

### 2-(4-biphenylyl)-1-{[(3S)-1-(3,3,3-trifluoropropanoyl)-3-pyrrolidinyl]methyl}-1H-imidazo[4,5-c]pyridine

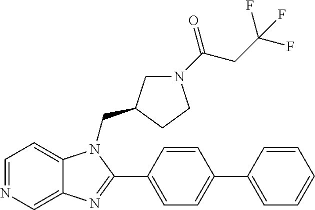

a) Following the procedure described in example 15d with 3,3,3-trifluoropropanoyl chloride provided the title compound as an off-white solid (39%). MS(ES)+ m/e 465 [M+H]⁺.

### Example 21

### (3S)-3-{[2-(4-biphenylyl)-1H-imidazo[4,5-c]pyridin-1-yl]methyl}-N-methyl-1-pyrrolidinecarboxamide

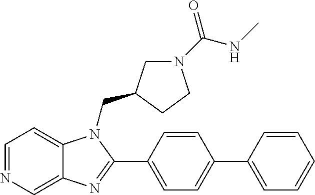

a) A solution of 2-(4-biphenylyl)-1-[(3R)-3-pyrrolidinylmethyl]-1H-imidazo[4,5-c]pyridine (0.11 mmol) in dichloromethane (2 mL) was treated with methyl isocyanate (0.12 mmol) and stirred at room temperature overnight. The reaction mixture was concentrated in vacuo and the residue was purified by reverse phase HPLC and concentrated in vacuo to provide the title product as a yellow trifluoroacetate salt (56 mg). MS(ES)+ m/e 412 [M+H]⁺.

### Example 22

### 2-[4-(1-benzofuran-5-yl)phenyl]-1-{[(3S)-1-(cyclopropylcarbonyl)-3-pyrrolidinyl]methyl}-1H-imidazol[4,5-c]pyridine

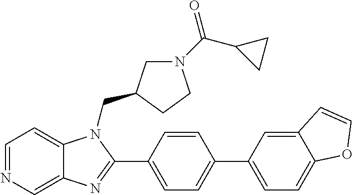

### a) 1-bromo-4-(2,2-dimethoxyethoxy)benzene

A mixture of 4-bromophenol (200 mmol), 2-bromo-1,1-dimethoxyethane (200 mmol), and potassium carbonate (200 mmol) in N,N-dimethylformamide (150 mL) was heated to 180° C. and stirred overnight. The reaction mixture was then cooled to room temperature and filtered. The filtrate was diluted with dichloromethane (200 mL) and the solution was washed with water (100 mL) and saturated brine (100 mL). The organic layer was dried over anhydrous sodium sulfate, filtered, and concentrated in vacuo to give a brown oil which was used directly in the next step without further purification.

### b) 5-bromobenzofuran

A mixture of 1-bromo-4-(2,2-dimethoxyethoxy)benzene (200 mmol) and polyphosphoric acid (30 mL) was heated to 100° C. and stirred overnight. The reaction mixture was cooled to room temperature and poured into aqueous sodium carbonate until the pH was ˜7 to 8. Then the mixture was extracted with dichloromethane (4×100 mL) and the organic layers were combined, washed with water (100 mL) and saturated brine (100 mL), dried over anhydrous sodium sulfate, filtered, and concentrated in vacuo. The mixture was distilled under reduced pressure (95° C., 5 mm Hg) to give a clear oil as the title product (25%). ¹H NMR (300 MHz, DMSO-d₆): δ 8.04 (s, 1H), 7.87 (s, 1H), 7.59 (d, J=8.4 Hz, 1H), 7.44 (d, J=8.4 Hz, 1H), 6.95 (s, 1H).

### c) 4-(benzofuran-5-yl)benzaldehyde

A mixture of 5-bromobenzofuran (51 mmol), 4-formylphenylboronic acid (61 mmol), Pd(dppf)Cl₂.CH₂Cl₂ (5.1 mmol), and potassium carbonate (51 mmol) in 1,4-dioxane (200 mL) was stirred at 100° C. under nitrogen gas atmosphere overnight. Then solvent was removed in vacuo and the residue was dissolved in dichloromethane (100 mL). The solution was washed with water (50 mL) and saturated brine (50 mL), and then the organic layer was dried over anhydrous sodium sulfate, filtered, and concentrated in vacuo. The residue was purified by flash chromatography (15:1 petroleum ether/ethyl acetate) to give the title product as a white solid (53%). ¹H NMR (300 MHz, DMSO-d₆): δ 10.07 (s, 1H), 8.09-7.93 (m, 6H), 7.73 (s, 2H), 7.05 (s, 1H).

### d) 1,1-dimethylethyl(3S)-3-{[(3-nitro-4-pyridinyl)amino]methyl}-1-pyrrolidinecarboxylate

A mixture of 4-chloro-3-nitropyridine (58.35 mmol), 1,1-dimethylethyl(3S)-3-(aminomethyl)-1-pyrrolidinecarboxylate (58.35 mmol), and triethylamine (58.35 mmol) in ethanol (150 mL) was stirred at 70° C. overnight. The reaction mixture was cooled to room temperature and the solvent was removed under reduced pressure. Purification of the residue by flash chromatography (3/1 ethyl acetate/petroleum ether and then 1% methanol/dichloromethane) afforded the title product as a yellow oil (77%). ¹H NMR (300 MHz, CDCl₃): δ 9.23 (s, 1H), 8.32 (d, J=6.0 Hz, 1H), 8.23-8.21 (m, 1H), 6.71 (d, J=6.3 Hz, 1H), 3.61-3.35 (m, 5H), 3.15-3.12 (m, 1H), 2.60-2.58 (m, 1H), 2.15-2.12 (m, 1H), 1.74-1.72 (m, 1H), 1.46 (s, 9H); MS(ES)+ m/e 323 [M+H]⁺.

### e) 3-nitro-N-[(3R)-3-pyrrolidinylmethyl]-4-pyridinamine

A solution of 1,1-dimethylethyl(3S)-3-{[(3-nitro-4-pyridinyl)amino]methyl}-1-pyrrolidinecarboxylate (44.75 mmol) in HCl/MeOH (3N, 200 mL) was stirred at 35° C. overnight. The solvent was then concentrated in vacuo to give the hydrochloride salt of the crude product (18.47 g) which was used directly in the next step without further purification. MS(ES)+ m/e 223 [M+H]⁺.

### f) N-{[(3S)-1-(cyclopropylcarbonyl)-3-pyrrolidinyl]methyl}-3-nitro-4-pyridinamine

To a −10° C. solution of crude 3-nitro-N-[(3R)-3-pyrrolidinylmethyl]-4-pyridinamine hydrochloride (18.47 g) in triethylamine (357.5 mmol) and dichloromethane (500 mL) was dropwise added cyclopropanecarbonyl chloride (58.35 mmol). After the addition, the mixture was stirred at room temperature for 2 h. The reaction was concentrated in vacuo and the residue was dissolved in ethyl acetate (100 mL). The mixture was washed with saturated brine (50 mL) and the organic layer was dried over anhydrous sodium sulfate, filtered, and concentrated in vacuo. Purification of the residue by flash chromatography (ethyl acetate, then 10% methanol/dichloromethane) gave the title product as yellow solid (62%). ¹H NMR (300 MHz, CDCl₃): δ 9.20 (d, J=3.6 Hz, 1H), 8.31 (t, J=5.1 Hz, 1H), 8.26-8.24 (m, 1H), 6.72 (t, J=5.7 Hz, 1H), 3.77-3.22 (m, 6H), 2.78-2.56 (m, 1H), 2.35-2.10 (m, 1H), 1.93-1.70 (m, 1H), 1.64-1.53 (m, 1H), 0.99-0.96 (m, 2H), 0.78-0.73 (m, 2H); MS(ES)+ m/e 291 [M+H]⁺.

### g) N⁴-{[(3S)-1-(cyclopropylcarbonyl)-3-pyrrolidinyl]methyl}-3,4-pyridinediamine

A mixture of N-{[(3S)-1-(cyclopropylcarbonyl)-3-pyrrolidinyl]methyl}-3-nitro-4-pyridinamine (36.2 mmol) and Pd/C (1 g, 10 wt %) in methanol (200 mL) was stirred at 30° C. overnight under a hydrogen atmosphere. The mixture was then filtered through Celite and the filtrate was concentrated in vacuo to give the crude product (9.4 g, 100%), which was used directly in the next step without further purification. ¹H NMR (300 MHz, CDCl₃): δ 7.90-7.81 (m, 2H), 6.46-6.42 (m, 1H), 4.90-4.83 (m, 1H), 3.96-3.23 (m, 8H), 2.70-2.51 (m, 1H), 2.24-2.04 (m, 1H), 1.89-1.16 (m, 2H), 0.98-0.97 (m, 2H), 0.80-0.76 (m, 2H); MS(ES)+ m/e 261 [M+H]⁺.

### h) 2-[4-(1-benzo furan-5-yl)phenyl]-1-{[(3S)-1-(cyclopropylcarbonyl)-3-pyrrolidinyl]methyl}-1H-imidazo[4,5-c]pyridine

A mixture of N⁴-{[(3S)-1-(cyclopropylcarbonyl)-3-pyrrolidinyl]methyl}-3,4-pyridinediamine (10 mmol) and 4-(benzofuran-5-yl)benzaldehyde (10 mmol) in n-butanol (120 mL) and acetic acid (6 mL) was stirred at 90° C. overnight. After the reaction was completed, the mixture was diluted with dichloromethane and washed with saturated aqueous sodium bicarbonate (50 mL) and saturated brine (50 mL). The organic layer was then dried over anhydrous sodium sulfate, filtered, and concentrated in vacuo. Purification of the residue by flash chromatography (1/20 methanol/dichloromethan) provided the title product as a yellow solid (56%). ¹H NMR (300 MHz, CDCl₃): δ 9.17 (s, 1H), 8.51-8.49 (m, 1H), 7.88 (s, 1H), 7.82 (d, J=2.7 Hz, 4H), 7.70 (d, J=1.8 Hz, 1H), 7.61 (s, 2H), 7.39 (d, J=5.4 Hz, 1H), 6.86 (d, J=1.8 Hz, 1H), 4.48-4.36 (m, 2H), 3.60-3.33 (m, 3H), 3.23-3.10 (m, 1H), 2.78-2.61 (m, 1H), 1.92-1.84 (m, 1H), 1.59-1.31 (m, 2H), 0.94-0.91 (m, 2H), 0.71-0.68 (m, 2H); MS(ES)+ m/e 463.2 [M+H]⁺.

### Example 23

### 1-{[(3S)-1-(cyclopropylcarbonyl)-3-pyrrolidinyl]methyl}-2-[4-(1H-indol-6-yl)phenyl]-1H-imidazo[4,5-c]pyridine

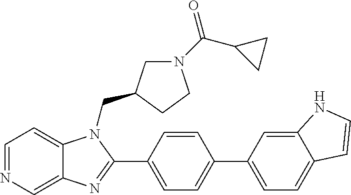

### a) 4-(1H-indol-6-yl)benzaldehyde

A mixture of 4-bromobenzaldehyde (2.70 mmol), indole-6-boronic acid (2.70 mmol), 1,1′-bis(diphenylphosphino)ferrocene-palladium(II)dichloride dichloromethane complex (0.135 mmol) and 2M potassium carbonate solution (4 mL, 8.00 mmol) in 1,4-dioxane (12 mL) was stirred at 100° C. for 1 h. The reaction was cooled to room temperature and diluted with ethyl acetate (50 mL) and water (50 mL). The aqueous layer was extracted with ethyl acetate (2×50 mL). The combined organic layer was washed with brine (20 mL), dried over magnesium sulfate, and concentrated in vacuo. Purification of the residue by flash chromatography (0-40% ethyl acetate/hexanes) provided the title compound as a yellow solid (97%). MS(ES)+ m/z 222.2 [M+H]⁺, 443.1 [2M+H]⁺.

### b) 1-{[(3S)-1-(cyclopropylcarbonyl)-3-pyrrolidinyl]methyl}-2-[4-(1H-indol-6-yl)phenyl]-1H-imidazo[4,5-c]pyridine

A yellow solution of N⁴-{[(3S)-1-(cyclopropylcarbonyl)-3-pyrrolidinyl]methyl}-3,4-pyridinediamine (0.34 mmol) and 4-(1H-indol-6-yl)benzaldehyde (0.34 mmol) in 1-butanol (2 mL) at 100° C. was stirred for 17 h. The reaction was monitored by LCMS. The temperature was increased and the reaction was stirred at 120° C. for 22 h. After this time, the reaction was stirred at 100° C. for an additional 72 h and the solution turned dark red. The reaction was cooled to room temperature and concentrated in vacuo to give a dark red foamy solid. Purification by flash chromatography (5% methanol/dichloromethane) provided the title compound as an orange foamy solid. Subsequent purification by reverse phase HPLC (65:35 300 mN aq ammonium formate:acetonitrile) provided the title compound as a yellow solid (39%). MS(ES)+ m/z 462.3 [M+H]⁺.

### Example 24

### 1-{[(3S)-1-(cyclopropylcarbonyl)-3-pyrrolidinyl]methyl}-2-[4-(1H-indazol-6-yl)phenyl]-1H-imidazo[4,5-c]pyridine

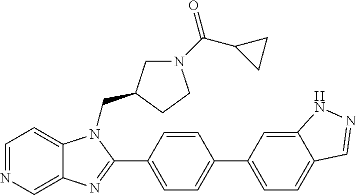

a) A solution of N⁴-{[(3S)-1-(cyclopropylcarbonyl)-3-pyrrolidinyl]methyl}-3,4-pyridinediamine (0.38 mmol) and 4-(1H-indazol-6-yl)benzaldehyde (0.31 mmol, example 47c) in N-methyl-2-pyrrolidone (4 mL) was heated at 130° C. overnight. The reaction mixture was cooled to room temperature and concentrated in vacuo. The residue was purified by reverse phase HPLC. Concentration of the product fractions afforded the title product as a solid (10%). MS(ES)+ m/e 463.2 [M+H]⁺.

### Example 25

### 1-{[(3S)-1-(cyclopropylcarbonyl)-3-pyrrolidinyl]methyl}-2-[4-(1H-indol-5-yl)phenyl]-1H-imidazo[4,5-c]pyridine

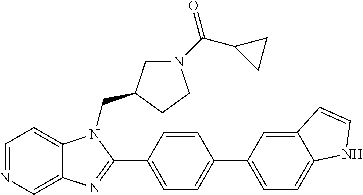

### a) 2-(4-bromophenyl)-1-{[(3S)-1-(cyclopropylcarbonyl)-3-pyrrolidinyl]methyl}-1H-imidazo[4,5-c]pyridine

A mixture of N⁴-{[(3S)-1-(cyclopropylcarbonyl)-3-pyrrolidinyl]methyl}-3,4-pyridinediamine (4.0 mmol), 4-bromobenzaldehyde (4.4 mmol), and acetic acid (2 mL) in n-butanol (60 mL) was stirred at 90° C. overnight. The reaction mixture was concentrated in vacuo and the residue was purified by flash chromatography (1/30 methanol/dichloromethane) to afford the title product (47%). ¹H NMR (300 MHz, CDCl₃): δ 9.15 (d, J=3.0 Hz, 1H), 8.52 (dd, J=2.4 Hz, 5.7 Hz, 1H), 7.74-7.70 (m, 2 H), 7.62-7.57 (m, 2H), 7.40 (d, J=5.7 Hz, 1H), 4.41-4.24 (m, 2H), 3.54-3.44 (m, 3H), 3.19-3.06 (m, 1H), 2.70-2.57 (m, 1H), 1.88-1.80 (m, 1H), 1.54-1.41 (m, 1H), 1.37-1.29 (m, 1H), 0.96-0.93 (m, 2H), 0.77-0.69 (m, 2H); MS(ES)+ m/e 425 [M+H]⁺.

### b) 1-{[(3S)-1-(cyclopropylcarbonyl)-3-pyrrolidinyl]methyl}-2-[4-(1H-indol-5-yl)phenyl]-1H-imidazo[4,5-c]pyridine

A mixture of 2-(4-bromophenyl)-1-{[(3S)-1-(cyclopropylcarbonyl)-3-pyrrolidinyl]methyl}-1H-imidazo[4,5-c]pyridine (1.8 mmol), 1H-indol-5-ylboronic acid (2.2 mmol), tetrakis(triphenylphosphine)palladium(0) (0.36 mmol), and sodium bicarbonate (5.4 mmol) in N,N-dimethylformamide (40 mL) was degassed and stirred at 100° C. overnight under a nitrogen atmosphere. The reaction mixture was poured into water and extracted with dichloromethane (100 mL×2). The combined organic layers were washed with saturated brine (50 mL), dried over sodium sulfate, filtered, and concentrated in vacuo. Purification of the residue by flash chromatography (1/60 methanol/dichloromethane) afforded the crude title product (336 mg, 40%), which was further purified by reverse phase HPLC to give clean product after neutralization of the product fractions (250 mg). ¹H NMR (300 MHz, DMSO-d₆): δ 11.22 (s, 1H), 9.01 (s, 1 H), 8.41 (dd, J=3.0 Hz, 5.4 Hz, 1H), 7.97 (s, 1H), 7.91-7.90 (m, 4H), 7.84 (d, J=5.4 Hz, 1H), 7.52 (s, 2H), 7.41 (t, J=2.7 Hz, 1H), 6.53 (s, 1H), 4.55-4.53 (m, 2H), 3.57-3.06 (m, 4H), 2.94-2.63 (m, 1H), 1.81-1.67 (m, 1H), 1.59-1.42 (m, 2H), 0.62-0.60 (m, 4H); MS(ES)+ m/e 462.1 [M+H]⁺.

### Example 26

### 3-{[(3S)-1-(cyclopropylcarbonyl)-3-pyrrolidinyl]methyl}-2-(4′-fluoro-4-biphenylyl)-3H-imidazo[4,5-b]pyridine

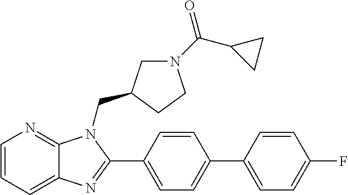

a) Following the procedure described in example 35b using 4′-fluoro-4-biphenylcarbaldehyde afforded the title product as a solid (25%). MS(ES)+ m/e 441.2 [M+H]⁺.

### Example 27

### 3-{[(3S)-1-(cyclopropylcarbonyl)-3-pyrrolidinyl]methyl}-2-[4′-(methyloxy)-4-biphenylyl]-3H-imidazo[4,5-b]pyridine

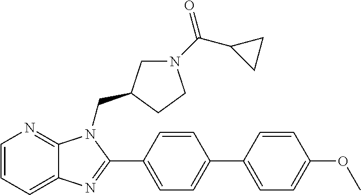

a) Following the procedure described in example 35b using 4′-(methyloxy)-4-biphenylcarbaldehyde afforded the title product as a solid (43%). MS(ES)+ m/e 453.2 [M+H]⁺.

### Example 28

### 3-{[(3S)-1-(cyclopropylcarbonyl)-3-pyrrolidinyl]methyl}-2-[4′-(ethyloxy)-4-biphenylyl]-3H-imidazo[4,5-b]pyridine

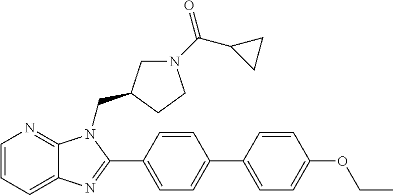

### a) 4′-(ethyloxy)-4-biphenylcarbaldehyde

Following the procedure described in example 29a using [4-(ethyloxy)phenyl]boronic acid gave the title product as a solid (72%).

### b) 3-{[(3S)-1-(cyclopropylcarbonyl)-3-pyrrolidinyl]methyl}-2-[4′-(ethyloxy)-4-biphenylyl]-3H-imidazo[4,5-b]pyridine

Following the procedure described in example 35b using 4′-(ethyloxy)-4-biphenylcarbaldehyde afforded the title product as a solid (41%). MS(ES)+ m/e 467.2 [M+H]⁺.

### Example 29

### 3-{[(3S)-1-(cyclopropylcarbonyl)-3-pyrrolidinyl]methyl}-2-(2′,4′-dimethyl-4-biphenylyl)-3H-imidazo[4,5-b]pyridine

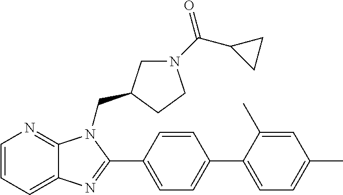

### a) 2′,4′-dimethyl-4-biphenylcarbaldehyde

A mixture of (2,4-dimethylphenyl)boronic acid (9.57 mmol), 4-bromobenzaldehyde (9.57 mmol), tetrakis(triphenylphosphine)palladium(0) (0.10 mmol), and potassium carbonate (26 mmol) in water (5 mL) and 1,4-dioxane (50 mL) was heated at 80° C. overnight. The reaction mixture was filtered and the filtrate was purified by reverse phase HPLC to afford the title product as a solid (81%).

### b) 3-{[(3S)-1-(cyclopropylcarbonyl)-3-pyrrolidinyl]methyl}-2-(2′,4′-dimethyl-4-biphenylyl)-3H-imidazo[4,5-b]pyridine

Following the procedure described in example 35b using 2′,4′-dimethyl-4-biphenylcarbaldehyde afforded the title product as a solid (36%). MS(ES)+ m/e 451.2 [M+H]⁺.

### Example 30

### 4′-(3-{[(3S)-1-(cyclopropylcarbonyl)-3-pyrrolidinyl]methyl}-3H-imidazo[4,5-b]pyridin-2-yl)-3-biphenylcarboxylic acid

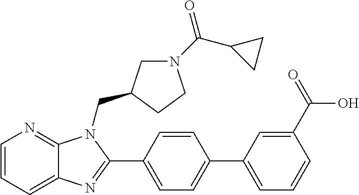

### a) 1,1-dimethylethyl(3S)-3-{[(3-nitro-2-pyridinyl)amino]methyl}-1-pyrrolidinecarboxylate

A solution of 2-chloro-3-nitropyridine (31.5 mmol), 1,1-dimethylethyl(3S)-3-(aminomethyl)-1-pyrrolidinecarboxylate (34.7 mmol), and triethylamine (47.3 mmol) in dimethylsulfoxide (50 mL) was heated at 100° C. for 5 h. The reaction mixture was diluted with water and extracted with dichloromethane. The combined organic layers were washed with brine, dried over sodium sulfate, filtered, and concentrated in vacuo to afford the crude title product.

### b) 3-nitro-N-[(3R)-3-pyrrolidinylmethyl]-2-pyridinamine

A solution of crude 1,1-dimethylethyl(3S)-3-{[(3-nitro-2-pyridinyl)amino]methyl}-1-pyrrolidinecarboxylate (31.5 mmol) in 2M HCl in methanol (50 mL) was stirred at room temperature for 12 h. Concentration in vacuo gave the crude title product as its hydrochloride salt.

### c) N-{[(3S)-1-(cyclopropylcarbonyl)-3-pyrrolidinyl]methyl}-3-nitro-2-pyridinamine

A 0° C. solution of crude 3-nitro-N-[(3R)-3-pyrrolidinylmethyl]-2-pyridinamine hydrochloride (20.3 mmol) and diisopropylethylamine (40.5 mmol) in acetonitrile (100 mL) was treated dropwise with cyclopropylcarbonyl chloride (30.4 mmol). The reaction was stirred at room temperature for 16 h, and then diluted with brine and extracted with dichloromethane. The combined organic layers were dried over sodium sulfate, filtered, and concentrated in vacuo. Purification of the residue by flash chromatography provided the title product (85%).

### d) N²-{[(3S)-1-(cyclopropylcarbonyl)-3-pyrrolidinyl]methyl}-2,3-pyridinediamine

A solution of N-{[(3S)-1-(cyclopropylcarbonyl)-3-pyrrolidinyl]methyl}-3-nitro-2-pyridinamine (24.1 mmol) in ethyl acetate (100 mL) was treated with 10% palladium on carbon (2 g) and hydrogenated at 40 psi for 5 h. The reaction mixture was filtered through Celite and concentrated in vacuo to afford the crude title compound as a solid (70%).

### e) 4′-(3-{[(3S)-1-(cyclopropylcarbonyl)-3-pyrrolidinyl]methyl}-3H-imidazo[4,5-b]pyridin-2-yl)-3-biphenylcarboxylic acid

A solution of N²-{[(3S)-1-(cyclopropylcarbonyl)-3-pyrrolidinyl]methyl}-2,3-pyridinediamine (0.46 mmol) and 4′-formyl-3-biphenylcarboxylic acid (0.46 mmol) in N-methyl-2-pyrrolidone (3 mL) was heated at 160° C. overnight. The reaction mixture was extracted with ethyl acetate, washed with water and brine, dried over sodium sulfate, filtered, and concentrated in vacuo. The residue was purified by reverse phase HPLC to provide the title product as a solid (56%). MS(ES)+ m/e 467.2 [M+H]⁺.

### Example 31

### 2-(3′-chloro-4-biphenylyl)-3-{[(3S)-1-(cyclopropylcarbonyl)-3-pyrrolidinyl]methyl}-3H-imidazo[4,5-b]pyridine

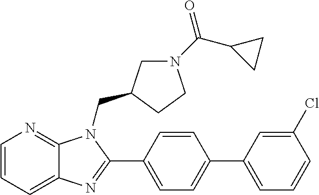

a) Following the procedure described in example 30e using 3′-chloro-4-biphenylcarbaldehyde afforded the title product as a solid (87%). MS(ES)+ m/e 457.2 [M+H]⁺.

### Example 32

### 2-(4′-chloro-4-biphenylyl)-3-{[(3S)-1-(cyclopropylcarbonyl)-3-pyrrolidinyl]methyl}-3H-imidazo[4,5-b]pyridine

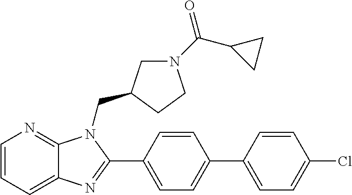

a) Following the procedure described in example 30e using 4′-chloro-4-biphenylcarbaldehyde afforded the title product as a solid (50%). MS(ES)+ m/e 457.2 [M+H]⁺.

### Example 33

### 2-(3′-chloro-4′-fluoro-4-biphenylyl)-3-{[(3S)-1-(cyclopropylcarbonyl)-3-pyrrolidinyl]methyl}-3H-imidazo[4,5-b]pyridine

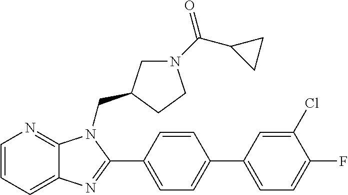

### a) 3′-chloro-4′-fluoro-4-biphenylcarbaldehyde

Following the procedure described in example 29a using (3-chloro-4-fluorophenyl)boronic acid gave the title product as a solid (87%).

### b) 2-(3′-chloro-4′-fluoro-4-biphenylyl)-3-{[(3S)-1-(cyclopropylcarbonyl)-3-pyrrolidinyl]methyl}-3H-imidazo[4,5-b]pyridine

Following the procedure described in example 35b using 3′-chloro-4′-fluoro-4-biphenylcarbaldehyde afforded the title product as a solid (27%). MS(ES)+ m/e 475.2 [M+H]⁺.

### Example 34

### 2-(4-biphenylyl)-3-{[(3S)-1-(cyclopropylcarbonyl)-3-pyrrolidinyl]methyl}-3H-imidazo[4,5-b]pyridine

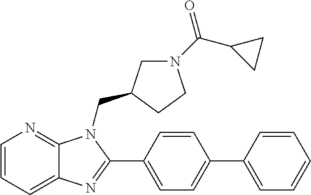

a) Following the procedure described in example 35b using 4-biphenylcarbaldehyde gave the title product as a solid (12%). MS(ES)+ m/e 423.2 [M+H]⁺.

### Example 35

### 3-{[(3S)-1-(cyclopropylcarbonyl)-3-pyrrolidinyl]methyl}-2-[4-(1H-indol-6-yl)phenyl]-3H-imidazo[4,5-b]pyridine

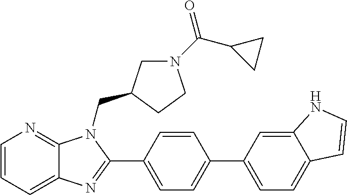

### a) 4-(1H-indol-6-yl)benzaldehyde

Following the procedure described in example 43b using 6-bromo-1H-indole (5.1 mmol) and 2M aq potassium carbonate at 80° C. and then purification by reverse phase HPLC in addition to preparative TLC gave the title product as a solid (75%).

### b) 3-{[(3S)-1-(cyclopropylcarbonyl)-3-pyrrolidinyl]methyl}-2-[4-(1H-indol-6-yl)phenyl]-3H-imidazo[4,5-b]pyridine

A solution of N²-{[(3S)-1-(cyclopropylcarbonyl)-3-pyrrolidinyl]methyl}-2,3-pyridinediamine (0.577 mmol) and 4-(1H-indol-6-yl)benzaldehyde (0.577 mmol) in N-methyl-2-pyrrolidone (4 mL) was heated at 150° C. for 3 days. The reaction mixture was extracted with ethyl acetate, washed with water and brine, dried over sodium sulfate, filtered, and concentrated in vacuo. The residue was purified by reverse phase HPLC to provide the title product as a solid (11%). MS(ES)+ m/e 462.2 [M+H]⁺.

### Example 36

### 2-[4-(1-benzofuran-5-yl)phenyl]-3-{[(3S)-1-(cyclopropylcarbonyl)-3-pyrrolidinyl]methyl}-3H-imidazo[4,5-b]pyridine

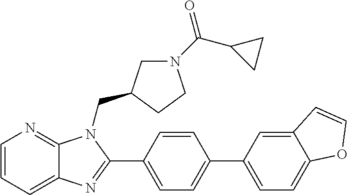

### a) 4-(1-benzofuran-5-yl)benzaldehyde

A mixture of 5-bromo-1-benzofuran (25.6 mmol), (4-formylphenyl)boronic acid (25.6 mmol), palladium(II) acetate (1.01 mmol), potassium carbonate (51.2 mmol), and dichloro[1,1′-bis(diphenylphosphino)ferrocene]palladium(II) (2.56 mmol) in water (20 mL) and 1,4-dioxane (50 mL) was heated at 80° C. overnight. The reaction mixture was extracted with ethyl acetate, washed with water and brine, dried over sodium sulfate, filtered, and concentrated in vacuo. The residue was purified by flash chromatography (10% ethyl acetate/petroleum ether) to afford the title product as a solid (35%).

### b) 2-[4-(1-benzo furan-5-yl)phenyl]-3-{[(3S)-1-(cyclopropylcarbonyl)-3-pyrrolidinyl]methyl}-3H-imidazo[4,5-b]pyridine

Following the procedure described in example 35b using 4-(1-benzofuran-5-yl)benzaldehyde afforded the title product as a solid (28%). MS(ES)+ m/e 463.2 [M+H]⁺.

### Example 37

### 6-chloro-3-{[(3S)-1-(cyclopropylcarbonyl)-3-pyrrolidinyl]methyl}-2-(2′-methyl-4-biphenylyl)-3H-imidazo[4,5-b]pyridine

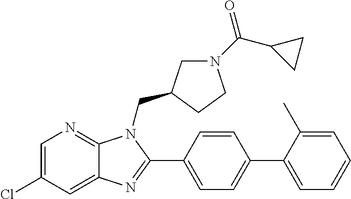

### a) 2′-methyl-4-biphenylcarbaldehyde

A mixture of (2-methylphenyl)boronic acid (1.30 mmol), 4-bromobenzaldehyde (1.08 mmol), and tetrakis(triphenylphosphine)palladium(0) (0.108 mmol) in 0.5M aq sodium carbonate (3 mL) and acetonitrile (3 mL) was heated at 90° C. overnight. The reaction mixture was diluted with water and extracted with (3×20 mL) ethyl acetate. The combined organic layers were dried over sodium sulfate, filtered, and concentrated in vacuo. Purification of the residue by flash chromatography (0-5% ethyl acetate/hexanes) gave the title product as a solid (74%). MS(ES)+ m/e 197 [M+H]⁺.

### b) 1,1-dimethylethyl(3S)-3-{[(5-chloro-3-nitro-2-pyridinyl)amino]methyl}-1-pyrrolidinecarboxylate

A solution of 2,5-dichloro-3-nitropyridine (1.56 mmol), 1,1-dimethylethyl(3S)-3-(aminomethyl)-1-pyrrolidinecarboxylate (1.56 mmol), and triethylamine (4.66 mmol) in ethanol (10 mL) was heated at 95° C. overnight. The reaction mixture was then concentrated in vacuo and the residue was purified by flash chromatography (20-40% ethyl acetate/hexanes) to afford the title product as a solid (41%). MS(ES)+ m/e 357 [M+H]⁺.

### c) 5-chloro-3-nitro-N-[(3R)-3-pyrrolidinylmethyl]-2-pyridinamine

A solution of 1,1-dimethylethyl(3S)-3-{[(5-chloro-3-nitro-2-pyridinyl)amino]methyl}-1-pyrrolidinecarboxylate (0.689 mmol) in 1,4-dioxane (3 mL) was treated with a 4M solution of HCl in 1,4-dioxane (5 mL) and stirred at room temperature for 1 h. The reaction was concentrated in vacuo and the remaining yellow solid was used without purification. MS(ES)+ m/e 257 [M+H]⁺.

### d) 5-chloro-N-{[(3S)-1-(cyclopropylcarbonyl)-3-pyrrolidinyl]methyl}-3-nitro-2-pyridinamine

A solution of 5-chloro-3-nitro-N-[(3R)-3-pyrrolidinylmethyl]-2-pyridinamine (5.49 mmol) and diisopropylethylamine (27.5 mmol) in dichloromethane (20 mL) was treated with cycloproylcarbonyl chloride (8.24 mmol) and stirred at room temperature for 3 h. The reaction was concentrated in vacuo and the residue was purified by flash chromatography (80-100% ethyl acetate/hexanes) to provide the title product as a solid (69%). MS(ES)+ m/e 325 [M+H]⁺.

### e) 5-chloro-N²-{[(3S)-1-(cyclopropylcarbonyl)-3-pyrrolidinyl]methyl}-2,3-pyridinediamine

A mixture of 5-chloro-N-{[(3S)-1-(cyclopropylcarbonyl)-3-pyrrolidinyl]methyl}-3-nitro-2-pyridinamine (2.99 mmol) and tin(II) chloride (14.9 mmol) in ethyl acetate (10 mL) was heated at 70° C. overnight. The reaction mixture was then cooled to room temperature, treated with 1N aq sodium hydroxide for 1 h, filtered, and partitioned. The organic layer was dried over sodium sulfate, filtered, and concentrated in vacuo to give the crude title compound as a solid (64%). This material was used without further purification. MS(ES)+ m/e 295 [M+H]⁺.

### f) 6-chloro-3-{[(3S)-1-(cyclopropylcarbonyl)-3-pyrrolidinyl]methyl}-2-(2′-methyl-4-biphenylyl)-3H-imidazo[4,5-b]pyridine

A solution of 5-chloro-N²-{[(3S)-1-(cyclopropylcarbonyl)-3-pyrrolidinyl]methyl}-2,3-pyridinediamine (0.109 mmol) and 2′-methyl-4-biphenylcarbaldehyde (0.109 mmol) in n-butanol (2 mL) was heated at 80° C. overnight. The reaction mixture was concentrated in vacuo and the residue was purified by flash chromatography (0-10% methanol/dichloromethane) to afford the title product as a solid (80%). MS(ES)+ m/e 471.2 [M+H]⁺.

### Example 38

### 6-chloro-3-{[(3S)-1-(cyclopropylcarbonyl)-3-pyrrolidinyl]methyl}-2-[3′-fluoro-4′-(methyloxy)-4-biphenylyl]-3H-imidazo[4,5-b]pyridine

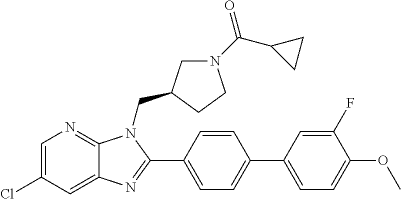

a) A solution of 5-chloro-N²-{[(3S)-1-(cyclopropylcarbonyl)-3-pyrrolidinyl]methyl}-2,3-pyridinediamine (0.109 mmol) and 3′-fluoro-4′-(methyloxy)-4-biphenylcarbaldehyde (0.109 mmol) in n-butanol (2 mL) was heated at 80° C. overnight. The reaction mixture was concentrated in vacuo and the residue was purified by reverse phase HPLC to afford the title product as a trifluoroacetate salt (40%). MS(ES)+ m/e 505.2 [M+H]⁺.

### Example 39

### 6-chloro-3-{[(3S)-1-(cyclopropylcarbonyl)-3-pyrrolidinyl]methyl}-2-(2′,4′-dimethyl-4-biphenylyl)-3H-imidazo[4,5-b]pyridine

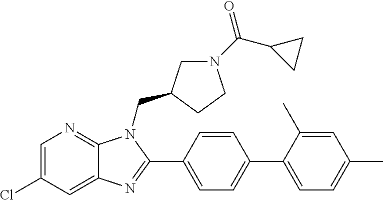

### a) 2-(4-bromophenyl)-6-chloro-3-{[(3S)-1-(cyclopropylcarbonyl)-3-pyrrolidinyl]methyl}-3H-imidazo[4,5-b]pyridine

A solution of 5-chloro-N²-{[(3S)-1-(cyclopropylcarbonyl)-3-pyrrolidinyl]methyl}-2,3-pyridinediamine (1.38 mmol) and 4-bromobenzaldehyde (1.38 mmol) in n-butanol (10 mL) was heated at 80° C. overnight. The reaction mixture was then cooled to room temperature and concentrated in vacuo. Purification of the residue by flash chromatography (20-40% ethyl acetate/hexanes) gave the product as a solid (63%). MS(ES)+ m/e 461 [M+H]⁺.

### b) 6-chloro-3-{[(3S)-1-(cyclopropylcarbonyl)-3-pyrrolidinyl]methyl}-2-(2′,4′-dimethyl-4-biphenylyl)-3H-imidazo[4,5-b]pyridine

A mixture of 2-(4-bromophenyl)-6-chloro-3-{[(3S)-1-(cyclopropylcarbonyl)-3-pyrrolidinyl]methyl}-3H-imidazo[4,5-b]pyridine (0.109 mmol), 2,4-dimethylphenylboronic acid (0.131 mmol), and tetrakis(triphenylphosphine)palladium(0) (0.0109 mmol) in 0.5M aq sodium carbonate (1 mL) and acetonitrile (2 mL) was heated at 90° C. overnight. The reaction mixture was cooled to room temperature and partitioned between water and ethyl acetate. The organic layers were dried over sodium sulfate, filtered, and concentrated in vacuo. Purification of the residue by flash chromatography (0-5% methanol/dichloromethane) gave the title product as a solid (28%). MS(ES)+ m/e 485.2 [M+H]⁺.

### Example 40

### 2-[4-(1-benzofuran-5-yl)phenyl]-6-chloro-3-{[(3S)-1-(cyclopropylcarbonyl)-3-pyrrolidinyl]methyl}-3H-imidazo[4,5-b]pyridine

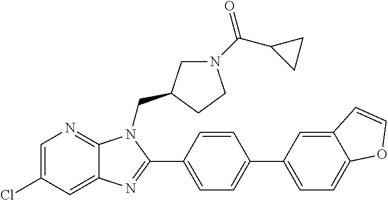

a) A mixture of 2-(4-bromophenyl)-6-chloro-3-{[(3S)-1-(cyclopropylcarbonyl)-3-pyrrolidinyl]methyl}-3H-imidazo[4,5-b]pyridine (0.109 mmol), 5-(4,4,5,5-tetramethyl-1,3,2-dioxaborolan-2-yl)-1-benzofuran (0.131 mmol), and tetrakis(triphenylphosphine)palladium(0) (0.0109 mmol) in 0.5M aq sodium carbonate (2 mL) and acetonitrile (2 mL) was heated at 90° C. overnight. The reaction mixture was cooled to room temperature and partitioned between water and ethyl acetate. The organic layers were dried over sodium sulfate, filtered, and concentrated in vacuo. Purification of the residue by flash chromatography (0-5% methanol/dichloromethane) gave the title product as a solid (17%). MS(ES)+ m/e 497.2 [M+H]⁺.

### Example 41

### 6-chloro-3-{[(3S)-1-(cyclopropylcarbonyl)-3-pyrrolidinyl]methyl}-2-[4-(1H-indazol-5-yl)phenyl]-3H-imidazo[4,5-b]pyridine

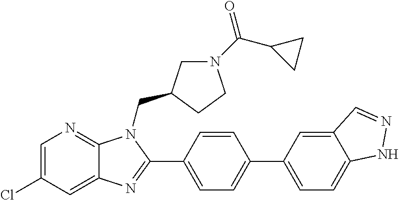

### a) 1,1-dimethylethyl 5-(4,4,5,5-tetramethyl-1,3,2-dioxaborolan-2-yl)-1H-indazole-1-carboxylate

A mixture of 1,1-dimethylethyl 5-bromo-1H-indazole-1-carboxylate (10.1 mmol), bis(pinacolato)diboron (11.1 mmol), palladium(II) acetate (1.01 mmol), dichloro[1,1′-bis(diphenylphosphino)ferrocene]palladium(II) (1.01 mmol), and potassium acetate (30.3 mmol) in triethylamine (1.5 mL) and 1,4-dioxane (60 mL) was heated at 110° C. overnight. The reaction mixture was concentrated in vacuo and the residue purified by flash chromatography (5% ethyl acetate/petroleum ether) to afford the title product as a yellow solid (74%).

### b) 6-chloro-3-{[(3S)-1-(cyclopropylcarbonyl)-3-pyrrolidinyl]methyl}-2-[4-(1H-indazol-5-yl)phenyl]-3H-imidazo[4,5-b]pyridine

A mixture of 2-(4-bromophenyl)-6-chloro-3-{[(3S)-1-(cyclopropylcarbonyl)-3-pyrrolidinyl]methyl}-3H-imidazo[4,5-b]pyridine (0.120 mmol), 1,1-dimethylethyl 5-(4,4,5,5-tetramethyl-1,3,2-dioxaborolan-2-yl)-1H-indazole-1-carboxylate (0.144 mmol), and tetrakis(triphenylphosphine)palladium(0) (0.012 mmol) in 0.5M aq sodium carbonate (2 mL) and acetonitrile (2 mL) was heated at 90° C. overnight. The reaction mixture was cooled to room temperature and partitioned between water and ethyl acetate. The organic layers were dried over sodium sulfate, filtered, and concentrated in vacuo. Purification of the residue by reverse phase HPLC (25-99% acetonitrile/water with 0.1% TFA) gave the title product as its trifluoroacetate salt (52%). MS(ES)+ m/e 497.2 [M+H]⁺.

### Example 42

### 2-[4-(1H-benzimidazol-5-yl)phenyl]-6-chloro-3-{[(351)-1-(cyclopropylcarbonyl)-3-pyrrolidinyl]methyl}-3H-imidazo[4,5-b]pyridine

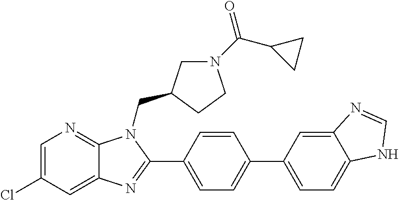

### a) 1,1-dimethylethyl 5-bromo-1H-benzimidazole-1-carboxylate

To a 0° C. solution of 5-bromo-1H-benzimidazole (10.2 mmol) in dichloromethane (30 mL) was dropwise added bis(1,1-dimethylethyl)dicarbonate (11.2 mmol) and triethylamine (15.2 mmol). The reaction mixture was then stirred at room temperature overnight. The mixture was washed with water (3×10 mL) and brine, then dried over sodium sulfate, filtered, and concentrated in vacuo to afford the crude product.

### b) 1,1-dimethylethyl 5-(4,4,5,5-tetramethyl-1,3,2-dioxaborolan-2-yl)-1H-benzimidazole-1-carboxylate

A mixture of 1,1-dimethylethyl 5-bromo-1H-benzimidazole-1-carboxylate (10.2 mmol), bis(pinacolato)diboron (11.2 mmol), palladium(II) acetate (1.02 mmol), dichloro[1,1′-bis(diphenylphosphino)ferrocene]palladium(II) (1.02 mmol), and potassium acetate (30.4 mmol) in triethylamine (2 mL) and 1,4-dioxane (50 mL) was heated at 110° C. overnight. The reaction mixture was concentrated in vacuo and the residue purified by flash chromatography (10% ethyl acetate/petroleum ether) to afford the title product as a solid (42%).

### c) 2-[4-(1H-benzimidazol-5-yl)phenyl]-6-chloro-3-{[(3S)-1-(cyclopropylcarbonyl)-3-pyrrolidinyl]methyl}-3H-imidazo[4,5-b]pyridine

A mixture of 2-(4-bromophenyl)-6-chloro-3-{[(3S)-1-(cyclopropylcarbonyl)-3-pyrrolidinyl]methyl}-3H-imidazo[4,5-b]pyridine (0.120 mmol), 1,1-dimethylethyl 5-(4,4,5,5-tetramethyl-1,3,2-dioxaborolan-2-yl)-1H-benzimidazole-1-carboxylate (0.144 mmol), and tetrakis(triphenylphosphine)palladium(0) (0.012 mmol) in 0.5M aq sodium carbonate (2 mL) and acetonitrile (2 mL) was heated at 90° C. overnight. The reaction mixture was cooled to room temperature and partitioned between water and ethyl acetate. The organic layers were dried over sodium sulfate, filtered, and concentrated in vacuo. Purification of the residue by reverse phase HPLC (25-99% acetonitrile/water with 0.1% TFA) gave the title product as its trifluoroacetate salt (32%). MS(ES)+ m/e 497.2 [M+H]⁺.

### Example 43

### 6-chloro-3-{[(3S)-1-(cyclopropylcarbonyl)-3-pyrrolidinyl]methyl}-2-[4-(1H-indazol-6-yl)phenyl]-3H-imidazo[4,5-b]pyridine

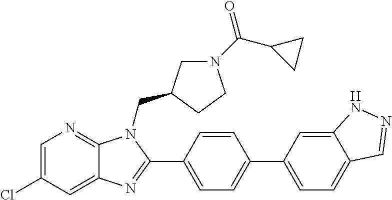

### a) 1,1-dimethylethyl 6-bromo-1H-indazole-1-carboxylate

To a 0° C. solution of 6-bromo-1H-indazole (20 mmol), triethylamine (4 mL), and 4-(dimethylamino)pyridine (4 mmol) in acetonitrile (100 mL) was dropwise added bis(1,1-dimethylethyl)dicarbonate (20 mmol) in acetonitrile. The reaction mixture was then stirred at room temperature overnight. The mixture was concentrated in vacuo and the residue was purified by flash chromatography (10% ethyl acetate/petroleum ether) to give the title product (63%).

### b) 4-(1H-indazol-6-yl)benzaldehyde

A mixture of 1,1-dimethylethyl 6-bromo-1H-indazole-1-carboxylate (0.675 mmol), (4-formylphenyl)boronic acid (0.675 mmol), and tetrakis(triphenylphosphine)palladium(0) (0.034 mmol), in 2M aq cesium carbonate (2 mL) and 1,4-dioxane (10 mL) was heated at 100° C. overnight. The reaction mixture was concentrated in vacuo and the residue purified by preparative TLC to afford the title product as a solid (33%).

### c) 6-chloro-3-{[(3S)-1-(cyclopropylcarbonyl)-3-pyrrolidinyl]methyl}-2-[4-(1H-indazol-6-yl)phenyl]-3H-imidazo[4,5-b]pyridine

A solution of 5-chloro-N²-{[(3S)-1-(cyclopropylcarbonyl)-3-pyrrolidinyl]methyl}-2,3-pyridinediamine (0.221 mmol) and 4-(1H-indazol-6-yl)benzaldehyde (0.221 mmol) in n-butanol (2 mL) was heated at 80° C. overnight. The reaction mixture was concentrated in vacuo and the residue was purified by reverse phase HPLC (25-99% acetonitrile/water with 0.1% TFA) to afford the title product as a trifluoroacetate salt (35%). MS(ES)+ m/e 497.2 [M+H]⁺.

### Example 44

### 8-[4-(1-benzofuran-5-yl)phenyl]-6-chloro-9-{[(3S)-1-(cyclopropylcarbonyl)-3-pyrrolidinyl]methyl}-9H-purine

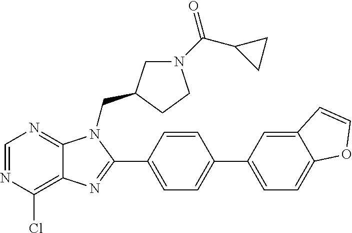

### a) 6-chloro-N-{[(3S)-1-(cyclopropylcarbonyl)-3-pyrrolidinyl]methyl}-5-nitro-4-pyrimidinamine

To a mixture of 4,6-dichloro-5-nitropyrimidine (1.03 mmol) in ethanol (8 mL) at room temperature was dropwise added a solution of {[(3S)-1-(cyclopropylcarbonyl)-3-pyrrolidinyl]methyl}amine (1.03 mmol) and triethylamine (1.03 mmol) in ethanol (2 mL). After 3 days, the reaction mixture was concentrated in vacuo and the residue was purified by flash chromatography (0-5% methanol/dichloromethane) to give the title product as a pale yellow solid (67%). MS(ES)+ m/e 326 [M+H]⁺.

### b) 6-chloro-N⁴-{[(3S)-1-(cyclopropylcarbonyl)-3-pyrrolidinyl]methyl}-4,5-pyrimidinediamine

To a vigorously stirred solution of 6-chloro-N-{[(3S)-1-(cyclopropylcarbonyl)-3-pyrrolidinyl]methyl}-5-nitro-4-pyrimidinamine (0.666 mmol) in acetic acid (20 mL) at room temperature was added powdered iron (6.66 mmol). The mixture was stirred overnight at room temperature and then concentrated in vacuo. The residue was partitioned between ethyl acetate and saturated aq sodium bicarbonate, and the aqueous layer was extracted with ethyl acetate until no chromophoric material remained. The combined organic layers were washed with saturated aq sodium bicarbonate and brine, dried over sodium sulfate, filtered, and concentrated in vacuo to give the title product as a yellow solid (87%). MS(ES)+ m/e 296 [M+H]⁺.

### c) 8-[4-(1-benzofuran-5-yl)phenyl]-6-chloro-9-{[(3S)-1-(cyclopropylcarbonyl)-3-pyrrolidinyl]methyl}-9H-purine

A solution of 6-chloro-N⁴-{[(3S)-1-(cyclopropylcarbonyl)-3-pyrrolidinyl]methyl}-4,5-pyrimidinediamine (0.507 mmol), 4-(1-benzofuran-5-yl)benzaldehyde (0.609 mmol), and acetic acid (0.873 mmol) in methanol (4 mL) was stirred at room temperature overnight. The heterogeneous solution was then concentrated in vacuo and concentrated from toluene twice. The residue was dissolved in ethanol (4 mL) and a solution of iron(III) chloride (0.507 mmol) in ethanol (2 mL) was added. The reaction mixture was refluxed for 1 h and then cooled to room temperature and concentrated in vacuo. Purification of the residue by reverse phase HPLC (20-70% acetonitrile/water with 0.1% TFA) gave the title product as a trifluoroacetate salt (39%). MS(ES)+ m/e 498.2 [M+H]⁺.

### Example 45

### 6-chloro-9-{[(3S)-1-(cyclopropylcarbonyl)-3-pyrrolidinyl]methyl}-8-(2′,4′-dichloro-4-biphenylyl)-9H-purine

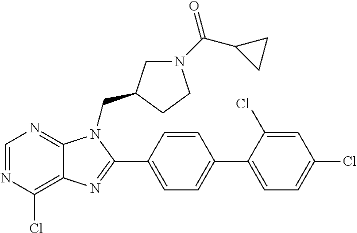

a) Following the procedure described in example 44c using 2′,4′-dichloro-4-biphenylcarbaldehyde provided the title product as a white trifluoroacetate salt (44%). MS(ES)+ m/e 526.1 [M+H]⁺.

### Example 46

### 8-(4-biphenylyl)-6-chloro-9-{[(3S)-1-(cyclopropylcarbonyl)-3-pyrrolidinyl]methyl}-9H-purine

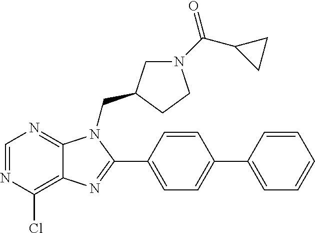

a) Following the procedure described in example 44c using 4-biphenylcarbaldehyde provided the title product as a white trifluoroacetate salt (50%). MS(ES)+ m/e 458.2 [M+H]⁺.

### Example 47

### 8-(4-biphenylyl)-9-{[(3S)-1-(cyclopropylcarbonyl)-3-pyrrolidinyl]methyl}-9H-purine

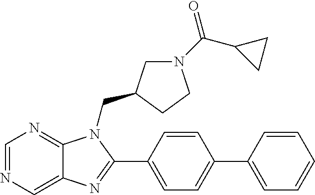

a) A mixture of 8-(4-biphenylyl)-6-chloro-9-{[(3S)-1-(cyclopropylcarbonyl)-3-pyrrolidinyl]methyl}-9H-purine (0.096 mmol) and 5% palladium on barium carbonate (0.019 mmol) in ethanol (10 mL) was reduced under a hydrogen atmosphere for 2 h. The hydrogen gas was removed and the reaction mixture was filtered through Celite and concentrated in vacuo. Purification of the residue by reverse phase HPLC (20-70% acetonitrile/water with 0.1% TFA) afforded the title product as a white trifluoroacetate salt (56%). MS(ES)+ m/e 424.2 [M+H]⁺.

### Example 48

### 8-[4-(1-benzofuran-5-yl)phenyl]-9-{[(3S)-1-(cyclopropylcarbonyl)-3-pyrrolidinyl]methyl}-9H-purine

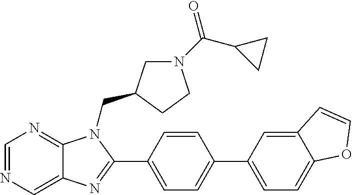

a) Following the procedure described in example 47a using 8-[4-(1-benzofuran-5-yl)phenyl]-6-chloro-9-{[(3S)-1-(cyclopropylcarbonyl)-3-pyrrolidinyl]methyl}-9H-purine afforded the title product as a white trifluoroacetate salt (60%). MS(ES)+ m/e 464.0 [M+H]⁺.

### Example 49

### 6-chloro-9-{[(3S)-1-(cyclopropylcarbonyl)-3-pyrrolidinyl]methyl}-8-(4′-fluoro-4-biphenylyl)-9H-purine

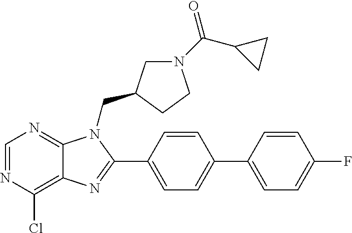

a) Following the procedure described in example 44c using 4′-fluoro-4-biphenylcarbaldehyde provided the title product as a white trifluoroacetate salt (50%). MS(ES)+ m/e 476.2 [M+H]⁺.

### Example 50

### 9-{[(3S)-1-(cyclopropylcarbonyl)-3-pyrrolidinyl]methyl}-8-(4′-fluoro-4-biphenylyl)-6-(4-methyl-1-piperazinyl)-9H-purine

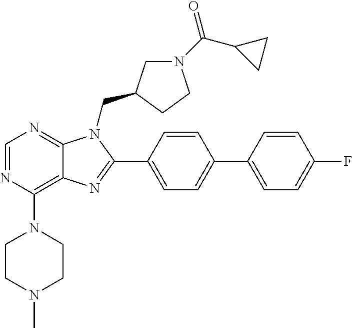

a) 6-Chloro-9-{[(3S)-1-(cyclopropylcarbonyl)-3-pyrrolidinyl]methyl}-8-(4′-fluoro-4-biphenylyl)-9H-purine (0.065 mmol) and 1-methylpiperazine (0.65 mmol) were irradiated in the microwave at 80° C. for 10 min. The reaction mixture was purified directly by reverse phase HPLC (1-99% acetonitrile/water with 0.1% TFA) to afford the title product as a white bistrifluoroacetate salt (63%). MS(ES)+ m/e 540.1 [M+H]⁺.

### Example 51

### 1-{[(3S)-1-(cyclopropylcarbonyl)-3-pyrrolidinyl]methyl}-2-[4′-(methyloxy)-4-biphenylyl]-1H-imidazo[4,5-b]pyridine

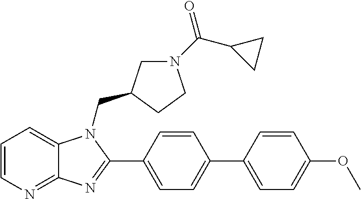

a) Following the procedure of example 1a using 4′-(methyloxy)-4-biphenylcarbaldehyde afforded the title product as a solid (36%). MS(ES)+ m/e 453.2 [M+H]⁺.

### Example 52

### 1-{[(3S)-1-(cyclopropylcarbonyl)-3-pyrrolidinyl]methyl}-2-[4-(1H-indol-5-yl)phenyl]-1H-imidazo[4,5-b]pyridine

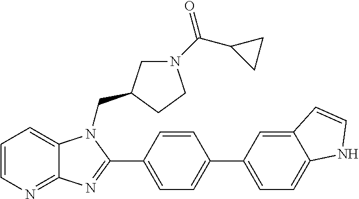

### a) 1,1-dimethylethyl(3S)-3-{[(2-nitro-3-pyridinyl)amino]methyl}-1-pyrrolidinecarboxylate

A solution of 3-chloro-2-nitropyridine (6.0 mmol) in dimethylsulfoxide (20 mL) was treated with 1,1-dimethylethyl(3S)-3-(aminomethyl)-1-pyrrolidinecarboxylate (6.0 mmol) and N,N-diisopropylethylamine (3.14 mL). The reaction was stirred under a stream of nitrogen at 70° C. overnight. The reaction mixture was diluted with water and extracted with dichloromethane. The organic phase was washed with brine, dried over magnesium sulfate, filtered, and concentrated in vacuo. Purification of the residue by flash chromatography (0-25% ethyl acetate/hexanes) gave the title compound as a yellow oil (400 mg, 20% yield). MS(ES)+ m/e 323.2 [M+H]⁺.

### b) 2-nitro-N-[(3R)-3-pyrrolidinylmethyl]-3-pyridinamine

A solution of 1,1-dimethylethyl(3S)-3-{[(2-nitro-3-pyridinyl)amino]methyl}-1-pyrrolidinecarboxylate (1.24 mmol) in methanol (10 mL) was treated with 4M HCl in dioxane (10 mL). The reaction was stirred at room temperature for 1 h. The reaction mixture was then concentrated in vacuo to dryness to afford the crude title compound as a yellow hydrochloride salt (quantitative). MS(ES)+ m/e 223.1 [M+H]⁺.

### c) N-{[(3S)-1-(cyclopropylcarbonyl)-3-pyrrolidinyl]methyl}-2-nitro-3-pyridinamine

A solution of 2-nitro-N-[(3R)-3-pyrrolidinylmethyl]-3-pyridinamine (1.24 mmol) and N,N-diisopropylethylamine (1.1 mL) in dichloromethane (5 mL) was stirred for 15 min at room temperature and then treated with a solution of cyclopropanecarbonyl chloride (1.49 mmol) in dichloromethane (5 mL). The reaction was stirred at room temperature for 1 h and then was diluted with saturated aq sodium bicarbonate and was stirred for 30 min. The organic phase was separated and the aqueous phase was extracted two additional times with dichloromethane. The combined organic layers were washed with brine, dried over magnesium sulfate, filtered, and concentrated in vacuo. The resulting crude yellow residue was used directly in the next step (quantitative). MS(ES)+m/e 291.0 [M+H]⁺.

### d) N³-{[(3S)-1-(cyclopropylcarbonyl)-3-pyrrolidinyl]methyl}-2,3-pyridinediamine

A solution of N-{[(3S)-1-(cyclopropylcarbonyl)-3-pyrrolidinyl]methyl}-2-nitro-3-pyridinamine (1.24 mmol) in ethanol (5 mL) and ethyl acetate (5 mL) was treated with 10% palladium on carbon (66 mg) and hydrogen gas from a balloon. After 2 h of stirring at room temperature, the reaction mixture was evacuated and backfilled with nitrogen. After confirming that the reduction had gone to completion, the mixture was filtered through Celite and concentrated in vacuo to afford the crude title compound as a gray oil (quantitative). MS(ES)+ m/e 261.1 [M+H]⁺.

### e) 2-(4-bromophenyl)-1-{[(3S)-1-(cyclopropylcarbonyl)-3-pyrrolidinyl]methyl}-1H-imidazo[4,5-b]pyridine

A solution of N3-{[(3S)-1-(cyclopropylcarbonyl)-3-pyrrolidinyl]methyl}-2,3-pyridinediamine (1.24 mmol) in 1-butanol (10 mL) was treated with 4-bromobenzyaldehyde (1.24 mmol). The reaction was stirred at 90° C. for 2 h. The reaction was diluted with brine (50 mL) and was extracted using ethyl acetate. The organic phase was washed with brine, dried over magnesium sulfate, filtered, and concentrated in vacuo. Purification of the residue by flash chromatography (0-10% methanol/ethyl acetate) afforded 410 mg of an oily material, which was then purified by chiral HPLC (Daicel Chiralpak IA column (4.6×150 mm) with a mobile phase of heptane:ethanol:isopropylamine (50:50:0.1), a flow rate of 1.0 mL/min, and UV detection at 254 nm gave a retention time of 5.7 min for the 2-(4-bromophenyl)-1-{[(3R)-1-(cyclopropylcarbonyl)-3-pyrrolidinyl]methyl}-1H-imidazo[4,5-b]pyridine and 7.8 min for the 2-(4-bromophenyl)-1-{[(3S)-1-(cyclopropylcarbonyl)-3-pyrrolidinyl]methyl}-1H-imidazo[4,5-b]pyridine) to afford the title compound as an off-white solid in >99% ee (370 mg, 70%). MS(ES)+ m/e 424.9/426.8 [M+H]⁺ (bromide isotope pattern).

### f) 1-{[(3S)-1-(cyclopropylcarbonyl)-3-pyrrolidinyl]methyl}-2-[4-(1H-indol-5-yl)phenyl]-1H-imidazo[4,5-b]pyridine

To as solution of 2-(4-bromophenyl)-1-{[(3S)-1-(cyclopropylcarbonyl)-3-pyrrolidinyl]methyl}-1H-imidazo[4,5-b]pyridine (0.235 mmol) in dioxane (2.5 mL) was added 5-(4,4,5,5-tetramethyl-1,3,2-dioxaborolan-2-yl)-1H-indole (0.26 mmol), dichloro[1,1′-bis(diphenylphosphino)ferrocene]palladium(II)-dichloromethane adduct (9.6 mg), and 2M aq potassium carbonate (0.353 mL). The reaction mixture was purged with nitrogen gas, sealed, and stirred overnight at 100° C. The reaction mixture was then cooled to room temperature and was diluted with water (50 mL). The aqueous layer was acidified to pH ˜7 using 1N aq HCl and was extracted using dichloromethane. The combined organic layers were dried over magnesium sulfate, filtered, and concentrated in vacuo. The brown residue was purified by reverse phase HPLC (LUNA C-18: 30×50 mm column; 0-35% acetonitrile w/0.1% TFA/water w/0.1% TFA). The product fractions were neutralized with the addition of saturated aq sodium bicarbonate, concentrated under reduced pressure, and extracted with dichloromethane. The combined organic layers were dried over magnesium sulfate, filtered, and concentrated in vacuo to afford the title compound as an off-white solid (35 mg, 31%). MS(ES)+ m/e 462.3 [M+H]⁺.

### Example 53

### 8-[4-(1-benzofuran-5-yl)phenyl]-9-{[(3S)-1-(cyclopropylcarbonyl)-3-pyrrolidinyl]methyl}-N-(1-methylethyl)-9H-purin-6-amine

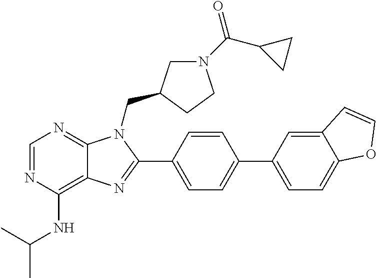

a) Following the procedure described in example 50a using isopropylamine provided the title product as a white trifluoroacetate salt (66%). MS(ES)+ m/e 521 [M+H]⁺.

### Example 54

### 1-{[(3S)-1-(cyclopropylcarbonyl)-3-pyrrolidinyl]methyl}-2-[4-(1H-pyrrolo[2,3-b]pyridin-5-yl)phenyl]-1H-imidazo[4,5-c]pyridine

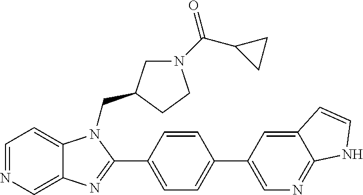

### a) 1,1-dimethylethyl(3S)-3-{[(3-nitro-4-pyridinyl)amino]methyl}-1-pyrrolidinecarboxylate

A solution of 4-chloro-3-nitropyridine (12.2 mmol) in ethanol (60 mL) was treated with 1,1-dimethylethyl(3S)-3-(amino methyl)-1-pyrrolidinecarboxylate (13.6 mmol) and triethylamine (5.68 mL). The reaction was stirred under a stream of nitrogen at 70° C. for 2 h. The reaction mixture was then concentrated in vacuo, dissolved in ethyl acetate (50 mL), and the organic layer was washed with brine, dried over magnesium sulfate, filtered, and concentrated in vacuo. Purification of the residue by flash chromatography (0-100% ethyl acetate/hexanes) gave the title compound as a yellow amorphous solid (3.74 g, 85% yield). MS(ES)+ m/e 323.2 [M+H]⁺.

### b) 3-nitro-N-[(3R)-3-pyrrolidinylmethyl]-4-pyridinamine

A solution of 1,1-dimethylethyl(3S)-3-{[(3-nitro-4-pyridinyl)amino]methyl}-1-pyrrolidinecarboxylate (11.6 mmol) in methanol (15 mL) was treated with 4M HCl in dioxane (60 mL). The reaction was stirred at room temperature for 1 h. The reaction mixture was then concentrated in vacuo to afford the crude title compound as a yellow hydrochloride salt (quantitative). MS(ES)+ m/e 223.2 [M+H]⁺.

### c) N-{[(3S)-1-(cyclopropylcarbonyl)-3-pyrrolidinyl]methyl}-3-nitro-4-pyridinamine

A solution of 3-nitro-N-[(3R)-3-pyrrolidinylmethyl]-4-pyridinamine (11.6 mmol) and N,N-diisopropylethylamine (10.1 mL) in dichloromethane (40 mL) was stirred at room temperature for 15 min and then treated with a solution of cyclopropanecarbonyl chloride (13.9 mmol) in dichloromethane (20 mL). The reaction was stirred at room temperature for 1 h and then was diluted with saturated aq sodium bicarbonate and was stirred for 30 min. The organic phase was separated and was washed with brine, dried over magnesium sulfate, filtered, and concentrated in vacuo. The resulting yellow residue was purified by flash chromatography (0-8% methanol/dichloromethane) to give the title compound as a yellow amorphous solid (3.1 g, 92% yield). MS(ES)+ m/e 291.0 [M+H]⁺.

### d) N⁴-{[(3S)-1-(cyclopropylcarbonyl)-3-pyrrolidinyl]methyl}-3,4-pyridinediamine

A solution of N-{[(3.5)-1-(cyclopropylcarbonyl)-3-pyrrolidinyl]methyl}-3-nitro-4-pyridinamine (10.7 mmol) in ethanol (50 mL) and ethyl acetate (50 mL) was treated with 10% palladium on carbon (570 mg) and hydrogen gas from a balloon. After 3 h of stirring at room temperature, the reaction mixture was evacuated and backfilled with nitrogen. After confirming that the reduction had gone to completion, the mixture was filtered through Celite and concentrated in vacuo to afford the crude title compound as a brown solid (2.8 grams, 80%). MS(ES)+ m/e 260.9 [M+H]⁺.

### e) 2-(4-bromophenyl)-1-{[(3S)-1-(cyclopropylcarbonyl)-3-pyrrolidinyl]methyl}-1H-imidazo[4,5-c]pyridine

A solution of N⁴-{[(3S)-1-(cyclopropylcarbonyl)-3-pyrrolidinyl]methyl}-3,4-pyridinediamine (10.3 mmol) in 1-butanol (75 mL) was treated with 4-bromobenzyaldehyde (10.3 mmol). The reaction was stirred at 105° C. overnight. Some starting material and dihydro-intermediate remained, so the temperature was increased to 120° C. for an additional 8 h. The reaction mixture was then concentrated in vacuo. Purification of the residue by flash chromatography (0-10% methanol/ethyl acetate) afforded 2.8 grams of a beige solid, which was then purified by chiral preparative SFC (Daicel Chiralcel OJ-H column (30×250 mm, 30° C.) with a mobile phase of 25% methanol:0.5% isopropylamine:75% liquid carbon dioxide, a flow rate of 70 mL/min, and UV detection at 240 nm gave a retention time of 5.0 min for the 2-(4-bromophenyl)-1-{[(3S)-1-(cyclopropylcarbonyl)-3-pyrrolidinyl]methyl}-1H-imidazo[4,5-c]pyridine and 6.3 min for the 2-(4-bromophenyl)-1-{[(3R)-1-(cyclopropylcarbonyl)-3-pyrrolidinyl]methyl}-1H-imidazo[4,5-c]pyridine) to afford the title compound as an off-white solid in >99% ee (1.6 grams, 37%). MS(ES)+ m/e 425/427 [M+H]⁺ (bromide isotope pattern).

### f) 1-{[(3S)-1-(cyclopropylcarbonyl)-3-pyrrolidinyl]methyl}-2-[4-(1H-pyrrolo[2,3-b]pyridin-5-yl)phenyl]-1H-imidazo[4,5-c]pyridine

To as solution of 2-(4-bromophenyl)-1-{[(3S)-1-(cyclopropylcarbonyl)-3-pyrrolidinyl]methyl}-1H-imidazo[4,5-c]pyridine (0.118 mmol) in dioxane (1 mL) was added 5-(4,4,5,5-tetramethyl-1,3,2-dioxaborolan-2-yl)-1H-pyrrolo[2,3-b]pyridine (0.123 mmol), dichloro[1,1′-bis(diphenylphosphino)ferrocene]palladium(II)-dichloromethane adduct (4.8 mg), and 2M aq potassium carbonate (0.176 mL). The reaction mixture was purged with nitrogen gas, sealed, and stirred for 3 h at 100° C. The reaction mixture was then cooled to room temperature and was diluted with water (50 mL). The aqueous layer was acidified to pH ˜7 using 1N aq HCl and was extracted using dichloromethane. The combined organic layers were dried over magnesium sulfate, filtered, and concentrated in vacuo. Purification of the brown residue by flash chromatography (0-10% methanol/dichloromethane) afforded the title compound as a beige solid (20 mg, 35%). MS(ES)+ m/e 463.4 [M+H]⁺.

### Example 55

### 1-{[(3S)-1-(cyclopropylcarbonyl)-3-pyrrolidinyl]methyl}-2-[4-(1H-indol-4-yl)phenyl]-1H-imidazo[4,5-c]pyridine

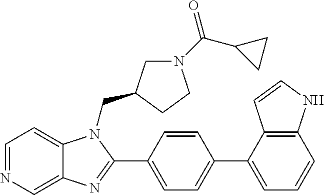

a) Following the procedure described in example 54f with 1H-indol-4-ylboronic acid provided the title product as a beige solid (42%). MS(ES)+ m/e 462.4 [M+H]⁺.

### Example 56

### 1-{[(3S)-1-(cyclopropylcarbonyl)-3-pyrrolidinyl]methyl}-2-[4-(1H-indol-7-yl)phenyl]-1H-imidazo[4,5-c]pyridine

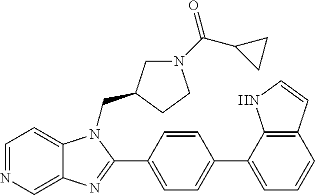

a) Following the procedure described in example 54f with 7-(4,4,5,5-tetramethyl-1,3,2-dioxaborolan-2-yl)-1H-indole provided the title product as a beige solid (60%). MS(ES)+ m/e 462.4 [M+H]⁺.

### Example 57

### 1-{[(3S)-1-(cyclopropylcarbonyl)-3-pyrrolidinyl]methyl}-2-[4-(1H-pyrrolo[3,2-b]pyridin-6-yl)phenyl]-1H-imidazo[4,5-c]pyridine

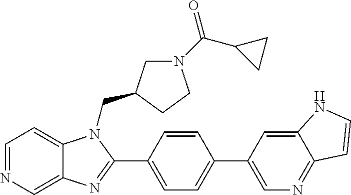

### a) 1-{[(3S)-1-(cyclopropylcarbonyl)-3-pyrrolidinyl]methyl}-2-[4-(4,4,5,5-tetramethyl-1,3,2-dioxaborolan-2-yl)phenyl]-1H-imidazo[4,5-c]pyridine

A solution of 2-(4-bromophenyl)-1-{[(3S)-1-(cyclopropylcarbonyl)-3-pyrrolidinyl]methyl}-1H-imidazo[4,5-c]pyridine (1.06 mmol) in anhydrous dioxane (10 mL) was treated with bis(pinocolato)diboron (1.06 mmol), dichloro[1,1′-bis(diphenylphosphino)ferrocene]palladium(II)-dichloromethane adduct (43 mg), and potassium acetate (3.17 mmol). The reaction mixturel was purged with nitrogen gas, sealed, and stirred for 3 h at 100° C. The black reaction mixture was then cooled to room temperature, diluted with water (50 mL), and extracted with ethyl acetate. The organic layer was washed with brine, dried over magnesium sulfate, filtered, and concentrated in vacuo to afford the title compound (as a mixture with the boronic acid) as a brown oil (296 mg, 59%). MS(ES)+ m/e 473.4 [M+H]⁺ (title product); (MS(ES)+ m/e 391.2 [M+H]⁺ (boronic acid).

### b) 1-{[(3S)-1-(cyclopropylcarbonyl)-3-pyrrolidinyl]methyl}-2-[4-(1H-pyrrolo[3,2-b]pyridin-6-yl)phenyl]-1H-imidazo[4,5-c]pyridine

A solution of 1-{[(3S)-1-(cyclopropylcarbonyl)-3-pyrrolidinyl]methyl}-2-[4-(4,4,5,5-tetramethyl-1,3,2-dioxaborolan-2-yl)phenyl]-1H-imidazo[4,5-c]pyridine (0.133 mmol) in dioxane (1 mL) was treated with 6-bromo-1H-pyrrolo[3,2-b]pyridine (0.127 mmol), dichloro[1,1′-bis(diphenylphosphino)ferrocene]palladium(II)-dichloromethane adduct (5.4 mg), and 2M aq potassium carbonate (0.200 mL). The reaction mixture was purged with nitrogen gas, sealed, and stirred at 100° C. for 3 h. The reaction mixture was then cooled to room temperature and was diluted with water (50 mL). The aqueous layer was acidified to pH ˜7 using 1N aq HCl and was extracted with dichloromethane. The organic layer was dried over magnesium sulfate, filtered, and concentrated in vacuo. The brown residue was purified by reverse phase HPLC (LUNA C-18: 30×50 mm column; 0-40% acetonitrile w/0.1% TFA/water w/0.1% TFA). The product fractions were neutralized with the addition of saturated aq sodium bicarbonate, concentrated under reduced pressure, and extracted with dichloromethane. The combined organic layers were dried over magnesium sulfate, filtered, and concentrated in vacuo to afford the title compound as a beige solid (10 mg, 15%). MS(ES)+ m/e 463.4 [M+H]⁺.

### Example 58

### 2-[4-(1,3-benzothiazol-5-yl)phenyl]-1-{[(3S)-1-(cyclopropylcarbonyl)-3-pyrrolidinyl]methyl}-1H-imidazo[4,5-c]pyridine

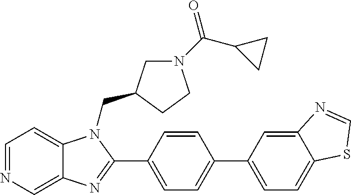

a) A solution of 5-bromo-1,3-benzothiazole (0.148 mmol) in anhydrous dioxane (1 mL) was treated with bis(pinocolato)diboron (0.141 mmol), dichloro[1,1′-bis(diphenylphosphino)ferrocene]palladium(II)-dichloromethane adduct (5.7 mg), and potassium acetate (0.423 mmol). The reaction mixture was purged with nitrogen gas, sealed, and stirred at 100° C. for 1 h. The black reaction mixture was then cooled to room temperature, and analysis by LC/MS confirmed the conversion of the starting material to its boronate ester. The solution was then treated with 2-(4-bromophenyl)-1-{[(3S)-1-(cyclopropylcarbonyl)-3-pyrrolidinyl]methyl}-1H-imidazo[4,5-c]pyridine (0.141 mmol), dichloro[1,1′-bis(diphenylphosphino)ferrocene]palladium(II)-dichloro methane adduct (5.7 mg), and 2M aq potassium carbonate (0.42 mmol). The reaction mixture was purged with nitrogen, sealed, and stirred at 100° C. overnight. The reaction mixture was cooled to room temperature and was diluted with water (50 mL). The aqueous layer was acidified to pH ˜7 using 1N aq HCl and was extracted with dichloromethane. The organic layer was dried over magnesium sulfate, filtered, and concentrated in vacuo. The brown residue was purified by reverse phase HPLC (LUNA C-18: 30×50 mm column; 0-30% acetonitrile w/0.1% TFA/water w/0.1% TFA). The product fractions were neutralized with the addition of saturated aq sodium bicarbonate, concentrated under reduced pressure, and extracted with dichloromethane. The combined organic layers were dried over magnesium sulfate, filtered, and concentrated in vacuo to afford the title compound as a beige solid (20 mg, 28%). MS(ES)+ m/e 480.1 [M+H]⁺.

### Example 59

### 1-{[(3S)-1-(cyclopropylcarbonyl)-3-pyrrolidinyl]methyl}-2-[4-(1H-indazol-4-yl)phenyl]-1H-imidazo[4,5-c]pyridine

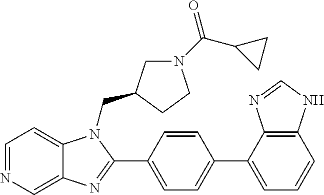

a) Following the procedure described in example 62a with 4-bromo-1H-indazole provided the title compound as a beige solid (12%). Reverse phase HPLC (LUNA C-18: 30×50 mm column; 0-30% acetonitrile w/0.1% TFA/water w/0.1% TFA) was utilized in purifying this compound. MS(ES)+ m/e 463.4 [M+H]⁺.

### Example 60

### 5-[4-(1-{[(3S)-1-(cyclopropylcarbonyl)-3-pyrrolidinyl]methyl}-1H-imidazo[4,5-c]pyridin-2-yl)phenyl]-1H-pyrazolo[3,4-b]pyridine

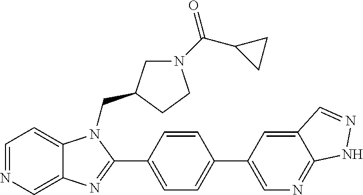

a) Following the procedure described in example 57b with 5-bromo-1H-pyrazolo[3,4-b]pyridine provided the title compound as a beige solid (8%). Both flash chromatography (0-10% methanol/dichloromethane) and reverse phase HPLC (LUNA C-18: 30×50 mm column; 0-30% acetonitrile w/0.1% TFA/water w/0.1% TFA) were utilized in purifying this compound. MS(ES)+ m/e 464.3 [M+H]⁺.

### Example 61

### 2-[4-(1H-benzimidazol-5-yl)phenyl]-1-{[(3S)-1-(cyclopropylcarbonyl)-3-pyrrolidinyl]methyl}-1H-imidazo[4,5-c]pyridine

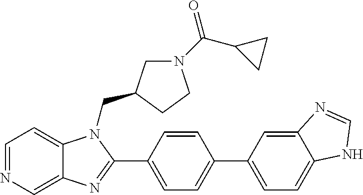

a) Following the procedure described in example 62a with 5-bromo-1H-benzimidazole provided the title compound as an off-white solid (6%). Reverse phase HPLC (LUNA C-18: 30×50 mm column; 0-25% acetonitrile w/0.1% TFA/water w/0.1% TFA) was utilized in purifying this compound. MS(ES)+ m/e 463.4 [M+H]⁺.

### Example 62

### 1-{[(3S)-1-(cyclopropylcarbonyl)-3-pyrrolidinyl]methyl}-2-[4-(1H-pyrrolo[2,3-b]pyridin-4-yl)phenyl]-1H-imidazo[4,5-c]pyridine

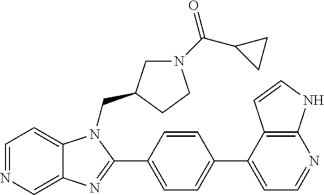

a) A solution of 2-(4-bromophenyl)-1-{[(3S)-1-(cyclopropylcarbonyl)-3-pyrrolidinyl]methyl}-1H-imidazo[4,5-c]pyridine (0.235 mmol) in dioxane (1.5 mL) was treated with bis(pinocolato)diboron (0.259 mmol), dichloro[1,1′-bis(diphenylphosphino)ferrocene]palladium(II)-dichloromethane adduct (9.6 mg), and potassium acetate (0.705 mmol). The reaction mixture was purged with nitrogen gas, sealed, and stirred at 100° C. for 2 h. The black reaction mixture was then cooled to room temperature, and analysis by LC/MS confirmed the conversion of the starting material to its boronate ester. The solution was then treated with 4-bromo-1H-pyrrolo[2,3-b]pyridine (0.212 mmol), dichloro[1,1′-bis(diphenylphosphino)ferrocene]palladium(II)-dichloromethane adduct (9.6 mg) and 2M aq potassium carbonate (0.705 mmol). The reaction mixture was purged with nitrogen, sealed, and stirred at 100° C. for 1 h. The reaction mixture was cooled to room temperature and was diluted with water (50 mL). The aqueous layer was acidified to pH ˜7 using 1N aq HCl and was extracted with dichloromethane. The organic layer was dried over magnesium sulfate, filtered, and concentrated in vacuo. The brown residue was purified by reverse phase HPLC (LUNA C-18: 30×50 mm column; 0-25% acetonitrile w/0.1% TFA/water w/0.1% TFA). The product fractions were neutralized with the addition of saturated aq sodium bicarbonate, concentrated under reduced pressure, and extracted with dichloromethane. The combined organic layers were dried over magnesium sulfate, filtered, and concentrated in vacuo to afford the title compound as a beige solid (46 mg, 40%). MS(ES)+ m/e 463.4 [M+H]⁺.

### Example 63

### 1-{[(3S)-1-(cyclopropylcarbonyl)-3-pyrrolidinyl]methyl}-2-(4-imidazo[1,2-c]pyridin-7-ylphenyl)-1H-imidazo[4,5-c]pyridine

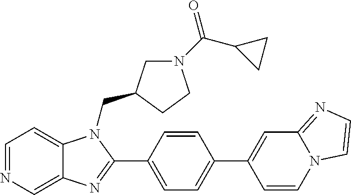

a) Following the procedure described in example 62a with 7-bromoimidazo[1,2-a]pyridine provided the title compound as a beige solid (34%). Reverse phase HPLC (LUNA C-18: 30×50 mm column; 0-30% acetonitrile w/0.1% TFA/water w/0.1% TFA) was utilized in purifying this compound. MS(ES)+ m/e 463.3 [M+H]⁺.

### Example 64

### 3-{[(3S)-1-(cyclopropylcarbonyl)-3-pyrrolidinyl]methyl}-2-[4-(1H-indol-6-yl)phenyl]-6-methyl-3H-imidazo[4,5-b]pyridine

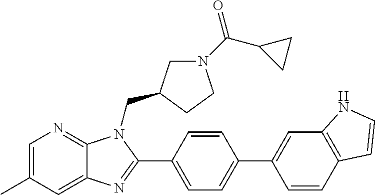

### a) 1,1-dimethylethyl(3S)-3-{[(5-methyl-3-nitro-2-pyridinyl)amino]methyl}-1-pyrrolidinecarboxylate

A mixture of 2-chloro-5-methyl-3-nitropyridine (1.73 mmol), 1,1-dimethylethyl (3S)-3-(aminomethyl)-1-pyrrolidinecarboxylate (1.91 mmol, ˜82% ee), and triethylamine (2.6 mmol) in dimethylsulfoxide (4 mL) was stirred at 100° C. overnight. After cooling to ambient temperature, the mixture was diluted with ethyl acetate (100 mL), washed with brine, dried over anhydrous sodium sulfate, and evaporated to dryness. The residue was then purified by silica gel chromatography (1:4 ethyl acetate:petroleum ether) to afford the desired product (322 mg, 55%). ¹H NMR (CDCl₃ w/TMS, 300 MHz): δ 1.46 (9H, s), 1.64-1.76 (1H, m), 2.00-2.10 (1H, m), 2.28 (3H, s), 2.53-2.65 (1H, m), 3.07-3.18 (1H, m), 3.27-3.67 (5H, m), 8.18 (1H, t, J=6.0 Hz), 8.24 (1H, s), 8.26 (1H, s); LCMS: m/e=337 [M+H]⁺.

### b) 5-methyl-3-nitro-N-[(3R)-3-pyrrolidinylmethyl]-2-pyridinamine

To a solution of 1,1-dimethylethyl(3S)-3-{[(5-methyl-3-nitro-2-pyridinyl)amino]methyl}-1-pyrrolidinecarboxylate (0.95 mmol) in dichloromethane (20 mL) was added trifluoroacetic acid (4.76 mmol) dropwise at 0° C. The reaction mixture was stirred at rt overnight. The solvent was then removed in vacuo to give the crude title product (225 mg) which used in the next step without purification. LCMS: m/e=237 [M+H]⁺.

### c) N-{[(3S)-1-(cyclopropylcarbonyl)-3-pyrrolidinyl]methyl}-5-methyl-3-nitro-2-pyridinamine

To a solution of 5-methyl-3-nitro-N-[(3R)-3-pyrrolidinylmethyl]-2-pyridinamine (0.95 mmol) and diisopropylethylamine (4.75 mmol) in dichloromethane (30 mL) was added cyclopropanecarbonyl chloride (1.43 mmol) dropwise at 0° C., and the mixture was stirred at rt overnight. The reaction mixture was diluted with dichloromethane (100 mL), washed with brine, dried over anhydrous sodium sulfate, and evaporated to dryness. The residue was then purified by silica gel chromatography (1:1 ethyl acetate:petroleum ether) to afford the title product (270 mg, 93%). ¹H NMR (CDCl₃ w/TMS, 300 MHz): δ 0.73-0.78 (2H, m), 0.97-1.02 (2H, m), 1.65-1.92 (1H, m), 2.05-2.20 (1H, m), 2.28 (3H, s), 2.55-2.79 (1H, m), 3.22-3.87 (7H, m), 8.18-8.28 (3H, m); LCMS: m/e=305 [M+H]⁺.

### d) N²-{[(3S)-1-(cyclopropylcarbonyl)-3-pyrrolidinyl]methyl}-5-methyl-2,3-pyridinediamine

A mixture of N-{[(3S)-1-(cyclopropylcarbonyl)-3-pyrrolidinyl]methyl}-5-methyl-3-nitro-2-pyridinamine (0.89 mmol) in methanol (20 mL) was hydrogenated over 10% Pd/C (13 mg) under a pressure of 30 psi hydrogen overnight. The mixture was then filtered through a pad of Celite and the filtrate was concentrated in vacuo to afford the title product (230 mg, 94%). LCMS: m/e=275 [M+H]⁺.

### e) 2-(4-bromophenyl)-3-{[(3S)-1-(cyclopropylcarbonyl)-3-pyrrolidinyl]methyl}-6-methyl-3H-imidazo[4,5-b]pyridine

A mixture of N²-{[(3S)-1-(cyclopropylcarbonyl)-3-pyrrolidinyl]methyl}-5-methyl-2,3-pyridinediamine (0.82 mmol), 4-bromobenzaldehyde (0.82 mmol), and acetic acid (0.5 mL) in n-butanol (20 mL) was stirred at 90° C. for 4 h. The solvent was removed in vacuo, and the residue was then purified by silica gel chromatography (1:1 ethyl acetate:petroleum ether) to afford the title product (220 mg, 61%). ¹H NMR (CDCl₃ w/TMS, 300 MHz): δ 0.67-0.75 (2H, m), 0.85-0.96 (2H, m), 1.33-1.52 (2H, m), 1.76-1.91 (1H, m), 2.50 (3H, s), 2.70-3.60 (5H, m), 4.43-4.50 (2H, m), 7.60-7.64 (2H, m), 7.67-7.72 (2H, m), 7.86-7.88 (1H, m), 8.23-8.25 (1H, m); LCMS: m/e=439 [M+H]⁺.

### f) 3-{[(3S)-1-(cyclopropylcarbonyl)-3-pyrrolidinyl]methyl}-2-[4-(1H-indol-6-yl)phenyl]-6-methyl-3H-imidazo[4,5-b]pyridine

A mixture of 2-(4-bromophenyl)-3-{[(3S)-1-(cyclopropylcarbonyl)-3-pyrrolidinyl]methyl}-6-methyl-3H-imidazo[4,5-b]pyridine (0.45 mmol), 1H-indol-6-ylboronic acid (0.5 mmol), Pd(PPh₃)₄ (0.045 mmol), and 1N aq NaHCO₃ (1.4 mL) in N,N-dimethylformamide (20 mL) was stirred at 100° C. overnight. The mixture was diluted with ethyl acetate (120 mL), washed with brine, dried over anhydrous sodium sulfate, filtered, and concentrated in vacuo. The residue was purified by silica gel chromatography (1:1 ethyl acetate:petroleum ether) to give the title product (150 mg, 69%, 82% ee). The product (105 mg) was purified by chiral HPLC (Lux 5u Cellulose-2, 60:40 ethanol:heptane) to afford the title product in 100% ee (74 mg). ¹H NMR (CDCl₃ w/TMS, 300 MHz): δ 0.66-0.69 (2H, m), 0.92-0.94 (2H, m), 1.38-1.62 (2H, m), 1.80-1.89 (1H, m), 2.51 (3H, s), 2.80-3.56 (5H, m), 4.51-4.59 (2H, m), 6.59 (1H, d, J=1.2 Hz) 7.29-7.89 (9H, m), 8.25 (1H, s), 8.85 (1H, s, br); MS(ES)+ m/e 476 [M+H]⁺.

### Example 65

### 3-{[(3S)-1-(cyclopropylcarbonyl)-3-pyrrolidinyl]methyl}-2-[4-(1H-indol-6-yl)phenyl]-5-(methyloxy)-3H-imidazo[4,5-b]pyridine

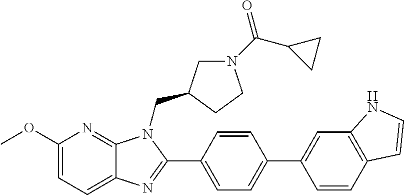

### a) N-{[(3S)-1-(cyclopropylcarbonyl)-3-pyrrolidinyl]methyl}-6-(methyloxy)-3-nitro-2-pyridinamine

A mixture of 2-chloro-6-(methyloxy)-3-nitropyridine (5.00 mmol) and {[(3S)-1-(cyclopropylcarbonyl)-3-pyrrolidinyl]methyl}amine (5.50 mmol, ˜80% ee) in triethylamine (1.4 mL) and ethanol (50 mL) was stirred at 80° C. overnight. The mixture was cooled to room temperature and the solvent was removed in vacuo. The residue was purified by silica gel chromatography (1:6 ethyl acetate:petroleum ether) to afford the desired product (1.4 g, 88%). ¹H NMR (CDCl₃ w/TMS, 300 MHz): δ 0.73-0.77 (2H, m), 0.98-1.01 (2H, m), 1.54-1.94 (2H, m), 2.06-2.26 (1H, m), 2.54-2.80 (1H, m), 3.22-3.88 (6H, m), 3.97 (3H, s), 6.08 (1H, d, J=9.0 Hz), 8.31 (1H, d, J=9.0 Hz), 8.82 (1H, s, br); LCMS: m/e=321 [M+H]⁺.

### b) N²-{[(3S)-1-(cyclopropylcarbonyl)-3-pyrrolidinyl]methyl}-6-(methyloxy)-2,3-pyridinediamine

A mixture of N-{[(3S)-1-(cyclopropylcarbonyl)-3-pyrrolidinyl]methyl}-6-(methyloxy)-3-nitro-2-pyridinamine (4.4 mmol) and 10% Pd/C (100 mg) in methanol (100 mL) was stirred at room temperature for 2 h under a balloon of hydrogen gas. The mixture was filtered through a pad of Celite and the filtrate was concentrated in vacuo to afford the desired product (1.21 g, 95%). LCMS: m/e=291 [M+H]⁺.

### c) 2-(4-bromophenyl)-3-{[(3S)-1-(cyclopropylcarbonyl)-3-pyrrolidinyl]methyl}-5-(methyloxy)-3H-imidazo[4,5-b]pyridine

A mixture of N²-{[(3S)-1-(cyclopropylcarbonyl)-3-pyrrolidinyl]methyl}-6-(methyloxy)-2,3-pyridinediamine (4.17 mmol) and 4-bromobenzaldehyde (4.17 mmol) in acetic acid (5 mL) and n-butanol (50 mL) was stirred at 90° C. for 3 h. The mixture was cooled to room temperature and the solvent was removed in vacuo. The residue was purified by silica gel chromatography (1:6 ethyl acetate:petroleum ether) to afford the desired product (1.17 g, 61%). ¹H NMR (CDCl₃ w/TMS, 300 MHz): δ 0.67-0.76 (2H, m), 0.92-0.98 (2H, m), 1.34-1.70 (2H, m), 1.82-1.96 (1H, m), 2.65-2.85 (1H, m), 3.18-3.66 (4H, m), 4.01 (3H, s), 4.39-4.45 (2H, m), 6.76 (1H, d, J=8.4 Hz), 7.60-7.71 (4H, m), 7.98 (1H, d, J=8.4 Hz); LCMS: m/e=455 [M+H]⁺.

### d) 3-{[(3S)-1-(cyclopropylcarbonyl)-3-pyrrolidinyl]methyl}-2-[4-(1H-indol-6-yl)phenyl]-5-(methyloxy)-3H-imidazo[4,5-b]pyridine

A mixture of 2-(4-bromophenyl)-3-{[(3S)-1-(cyclopropylcarbonyl)-3-pyrrolidinyl]methyl}-5-(methyloxy)-3H-imidazo[4,5-b]pyridine (0.793 mmol), 1H-indol-6-ylboronic acid (0.795 mmol) and Pd(PPh₃)₄ (0.079 mmol) in 1N aq NaHCO₃ (0.79 mL) and N,N-dimethylformamide (20 mL) was stirred at 80° C. overnight. The mixture was cooled to room temperature, poured into water, and extracted with ethyl acetate. The organic layer was washed with brine, dried over anhydrous sodium sulfate, filtered, and concentrated in vacuo. The residue was purified by Prep-TLC (ethyl acetate) to afford the desired product (110 mg, 28%, 80% ee). The product (92 mg) was purified by chiral HPLC (Chiralpak AD-H, 50:50 ethanol:heptane) to afford the title product in >98% ee (60 mg). ¹H NMR (CDCl₃ w/TMS, 300 MHz): δ 0.65-0.73 (2H, m), 0.86-0.96 (2H, m), 1.26-1.97 (3H, m), 2.70-2.93 (1H, m), 3.27-3.64 (4H, m), 4.02 (3H, s), 4.47-4.54 (2H, m), 6.58 (1H, s), 6.77-6.80 (1H, m), 7.27-7.29 (1H, m), 7.40-7.44 (1H, m), 7.64-7.82 (6H, m), 8.05 (1H, d, J=8.7 Hz), 8.69 (1H, s, br); MS(ES)+ m/e 492 [M+H]⁺.

### Biological Assays

FAS assay
FAS activity was measured through one of the two following assays.

### Assay #1:

Inhibition of FAS activity can be measured based on the detection of residual NADPH substrate after the FAS assay is quenched. This assay is run as a 10 μL endpoint assay in 384-well format, where the reaction contains 20 μM malonyl-CoA, 2 μM acetyl-CoA, 30 μM NADPH and 40 nM FAS in 50 mM sodium phosphate, pH 7.0. The assay is run by sequentially dispensing 5 μl of a malonyl-CoA solution, then enzyme solution (containing the acetyl-CoA, and NADPH) into a black, low volume assay plate (Greiner 784076) pre-dispensed with 100 mL compound solutions in DMSO. The reaction is incubated at ambient temperature for 60 minutes, then quenched with 5 μL of a developing solution composed of 90 μM resazurin, 0.3 IU/ml diaphorase in 50 mM sodium phosphate, pH 7.0. The developed reaction is read on a Molecular Devices Analyst or Acquest (or equivalent) plate reader using a 530 nm excitation wavelength filter, a 580 nm emission filter, and 561 nm dichroic filter. The test compounds are prepared in neat DMSO at a concentration of 10 mM. For inhibition curves, compounds are diluted using a three fold serial dilution and tested at 11 concentrations (e.g. 25 μM-0.42 nM). Curves are analysed using Activityl)ase and XLfit, and results are expressed as pIC50 values.

### Assay #2:

Inhibition of FAS can also be quantified based on the detection of the CoA products with a thio-reactive coumarin dye. This assay is run as a 10 μL endpoint assay in 384-well format, where the reaction contains 20 μM malonyl-CoA, 20 μM acetyl-CoA, 40 μM NADPH and 2 nM FAS in 50 mM sodium phosphate, pH 7.0, and 0.04% Tween-20. The assay is run by adding 5 μL enzyme solution to a black, low volume assay plate (Greiner 784076) pre-dispensed with 100 nl compound solutions in DMSO. After 30 minutes, 5 μL substrate is added, and the reaction incubated at ambient temperature for an additional 60 minutes. The reaction is then quenched with 10 μL of 6M guanidine-HCl containing 50 μM CPM (7-diethylamino-3-(4′-maleimidylphenyl)-4-methylcoumarin (CPM; thio-reactive dye), and incubated for 30 minutes. The plate is read on an Envision (PerkinElmer) or equivalent plate reader using a 380 nm excitation wavelength filter, and a 486 nm emission filter. Data fitting and compound preparations are done as described above.

### Lipogenesis Assay

Cultured primary human pre-adipocytes (Zen-Bio, Cat# ASC062801) are plated at confluence (3×10⁴ cells/well) in 96-well plates (Costar, Cat#3598) coated with 0.2% gelatin (Sigma, Cat# G-6650) in DMEM/F12 medium (InvitroGen Cat#11330-032) supplemented with 10% heat inactivated fetal bovine serum (InvitroGen, Cat#16000-044). The following day (day 1) the cell differentiation is induced by replacing the seeding medium with the differentiation medium composed of DMEM/F12 medium supplemented with 10% heat inactivated fetal bovine serum, 200 μM 3-isobutyl-1-methylxanthine (Sigma, Cat# I-5879), 20 nM dexamethasone (Sigma, Cat# D-8893), 20 nM GW1929 (Sigma, Cat# G5668) and 20 nM insulin (InvitroGen, Cat#03-0110SA). On day 7, differentiation medium is replaced by the re-feed medium made of DMEM/F12 supplemented with 10% heat inactivated serum and 20 nM insulin. The appropriate concentration of tested compounds and controls are added into this medium at that time. On day 12, the relative amount of cellular triglyceride is estimated by using a Trinder kit (Sigma, Cat# TR0100). Re-feed medium is aspirated and cells are washed with PBS (InvitroGen, Cat#14190-144) and the assay is performed according the kit manufacturer protocol. Briefly, reconstituted solutions A and B are mixed with 0.01% digitonin (Sigma, Cat# D-5628) prior to performing the assay and added onto the cells; plates are incubated at 37° C. for one hour. The absorbance is read at 540 nm. The data is first normalized using the following equation: 100*((UNK−Control 1)/(Control 2−Control 1)) where Control 1 is the Robust Mean of the 0% response control and Control 2 is the Robust Mean of the 100% response control. When multiple dilutions of compounds are tested, pXC50 are calculated from curves using the 4-parameter curve fitting with the following equation: y=(a−d)/(1+(s/c)̂b)+d and with IRLS (Iterative Re-weighted Least Squares) algorithms to weight outliers (Mosteller, F. & Tukey J. W. (1977) Data Analysis and Regression, pp 353-365, Addison-Wesley).

### Biological Data

Exemplified compounds of the present invention were tested according to the above assays and were found to be inhibitors of FAS. The IC₅₀ values ranged from about 1 nM to about 10 μM. The IC₅₀ values of the more active compounds range from about 1 nM to about 200 nM. The most active compounds are under 10 nM.

The compound of Example 2 was tested generally according to the assays described herein and in at least one experimental run exhibited an IC₅₀ value equal to 0.063 μM.

The compound of Example 19 was tested generally according to the assays described herein and in at least one experimental run exhibited an IC₅₀ value equal to 1.058 μM.

The compound of Example 22 was tested generally according to the assays described herein and in at least one experimental run exhibited an IC₅₀ value equal to 0.06 μM.

The compound of Example 25 was tested generally according to the assays described herein and in at least one experimental run exhibited an IC₅₀ value equal to 0.019 μM.

The compound of Example 27 was tested generally according to the assays described herein and in at least one experimental run exhibited an IC₅₀ value equal to 0.045 μM.

The compound of Example 35 was tested generally according to the assays described herein and in at least one experimental run exhibited an IC₅₀ value equal to 0.009 μM.

The compound of Example 46 was tested generally according to the assays described herein and in at least one experimental run exhibited an IC₅₀ value equal to 0.129 μM.

The compound of Example 54 was tested generally according to the assays described herein and in at least one experimental run exhibited an IC₅₀ value equal to 0.142 μM

The compound of Example 55 was tested generally according to the assays described herein and in at least one experimental run exhibited an IC₅₀ value equal to 0.013 μM.

The compound of Example 58 was tested generally according to the assays described herein and in at least one experimental run exhibited an IC₅₀ value equal to 0.010 μM.

1. A compound of the Formula (I), 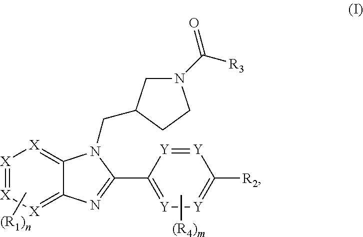
wherein,
each R₁ is independently selected from the group consisting of: halogen, C1-6alkyl, alkoxy, hydroxyl, amino, substituted amino, alkylsulfonyl, C4-7heterocycloalkyl, cyano, and —C(O)NRₐR_{b}, in which Rₐ and R_{b} are hydrogen, C1-6alkyl, C3-7cycloalkyl, or together Rₐ and R_{b} form a C4-7heterocycloalkyl;
R₂ is selected from the group consisting of: aryl and heteroaryl, in which adjacent substituents in said aryl or heteroaryl together may form an additional five or six membered ring which contains 0-2 hetero atoms;
R₃ is selected from the group consisting of: amino, alkylamino, dialkylamino, C1-6alkyl, —OC1-6alkyl, and C3-7cycloalkyl;
R₄ is selected from the group consisting of: C1-6alkyl, alkoxy, hydroxyl, and halogen;
Y and X are C or N;
n is 0-3; and
m is 0-4;
or a pharmaceutically acceptable salt thereof;
with the proviso that at least one but no more than two X's are N and at least two Y's are C.

2. A compound of claim 1, wherein said compound is represented by Formula (I)(A), as shown below 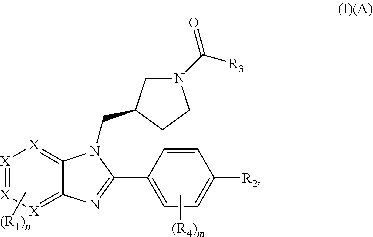
wherein,
each R₁ is independently selected from the group consisting of: C1-6alkyl, alkoxy, hydroxyl, cyano, alkylsulfonyl, halogen, amino, alkylamino, dialkylamino, heterocycloalkyl and —C(O)NRₐR_{b}, in which Rₐ and R_{b} are hydrogen, C1-6alkyl, C3-7cycloalkyl, or together Rₐ and R_{b} form a C3-7heterocycloalkyl
R₂ is selected from the group consisting of: aryl and heteroaryl, in which adjacent substituents in said aryl or heteroaryl together may form an additional five or six membered ring which contains 0-2 hetero atoms;
R₃ is selected from the group consisting of: amino, alkylamino, dialkylamino, —OC1-6alkyl, C1-6alkyl and C3-7cycloalkyl;
R₄ is selected from the group consisting of: C1-6alkyl, alkoxy, hydroxyl, and halogen;
X is C or N;
n is 0-3;
m is 0-3;
or a pharmaceutically acceptable salt thereof;
with the proviso that at least one but no more than two X's are N.

3. A compound of claim 1, wherein R₃ is cyclopropyl.

4. A compound of claim 1, wherein n is 0-2 and m is 0.

5. A compound of claim 1, wherein n is 0-2 and m is 1.

6. A compound of claim 1, wherein R₁ is halogen, C1-3alkyl, or —C(O)NRₐR_{b} as defined above.

7. A compound of claim 1, wherein R₂ is heteroaryl.

8. A compound of claim 1, wherein R₂ is aryl.

9. A pharmaceutical composition comprising a compound according to claim 1 and a pharmaceutically acceptable carrier.

10. A method of treating cancer which comprises administering to a human in need thereof an effective amount of a compound as described in claim 1.

11. A method of claim 10 wherein the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, renal, ovarian, pancreatic, prostate, sarcoma, osteosarcoma, giant cell tumor of bone and thyroid.

12. A compound of claim 1, which is represented by Formula (I)(B) 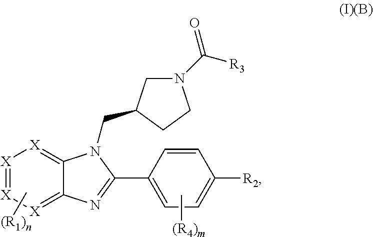
wherein R₂ is heteroaryl.

13. A compound of claim 12, wherein R₂ is pyrrolopyridinyl, imidazopyridinyl, benzimidazolyl, benzothiazolyl, benzofuranyl or indolyl.

14. A method of treating cancer comprising administering to a human in need thereof an effective amount of a compound of claim 1, or a pharmaceutically acceptable salt thereof, according to claim 1, in a pharmaceutically acceptable composition.

15. A method of claim 14, wherein the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone and thyroid.
